(19) United States
(12) Patent Application Publication
(10) Pub. No.: US 2025/0241254 A1
(43) Pub. Date: Jul. 31, 2025

(54) NOVEL GENETIC LOCI ASSOCIATED WITH DISEASE RESISTANCE IN SOYBEANS

(71) Applicant: Syngenta Crop Protection AG, Basel (CH)

(72) Inventors: Qingli Liu, Durham, NC (US); Robert Arthur Dietrich, Durham, NC (US); Thomas Joseph Curley, Jr., Durham, NC (US); John Daniel HIPSKIND, Durham, NC (US); Becky Welsh Breitinger, Durham, NC (US); John Luther Dawson, Durham, NC (US)

(73) Assignee: Syngenta Crop Protection AG, Basel (CH)

(21) Appl. No.: 18/728,946

(22) PCT Filed: Jan. 10, 2023
(86) PCT No.: PCT/US2023/060373

§ 371 Date: Jul. 15, 2024

Related U.S. Application Data
(60) Provisional application No. 63/299,566, filed on Jan. 14, 2022.

(51) Int. Cl. A01H 1/04; A01H 1/00; C12Q 1/6806; C12Q 1/6895

(52) U.S. Cl. CPC A01H 1/045; A01H 1/1255; C12Q 1/6806; C12Q 1/6895; C12Q 2600/13

(57) ABSTRACT

The present invention relates to methods and compositions for identifying, selecting and/or producing a disease resistant soybean plant or germplasm using markers, genes and chromosomal intervals derived from Glycine canescens PI446934, or a progeny thereof. A soybean plant or germplasm that has been identified, selected and/or produced by any of the methods of the present invention is also provided. Embodiments of soybean seeds, plants and germplasms are also provided that are resistant to Asian Soy Rust.

### RELATED APPLICATIONS

This application claims priority to U.S. Provisional Patent Application No. 63/299,566 filed 14 January, 2022, the content of which is incorporated by reference herein in its entirety.

### FIELD OF THE INVENTION

The present invention relates to compositions and methods for identifying, selecting and producing enhanced disease and/or pathogen resistant soybean plants.

### SEQUENCE LISTING

This application is accompanied by a sequence listing entitled 81545.xml, created Jan. 5, 2023, which is approximately 936 KB in size. This sequence listing filed herewith via EFS-Web in compliance with 37 C.F.R. § 1.831-1.835, is incorporated by reference into the specification in its entirety.

### BACKGROUND

Plant pathogens are known to cause considerable damage to important crops, resulting in significant agricultural losses with widespread consequences for both the food supply and other industries that rely on plant materials. As such, there is a long felt need to reduce the incidence and/or impact of agricultural pathogens on crop production.

Several pathogens have been associated with damage to soybeans, which individually and collectively have the potential to cause significant yield losses in the United States and throughout the world. Exemplary pathogens include, but are not limited to fungi (e.g., genus Phytophthora and Asian Soybean rust Phakopsora pahyrhizi), nematodes (e.g., genus Meloidogyne, particularly, Meloidogyne javanica), and soybean stem canker. Given the significant threat to global food supplies that these pathogens present and the time and expense associated with treating soybean crops to prevent loss, new methods for producing pathogen resistant soybean cultivars are needed. What is needed is novel resistance genes (herein, “R-Genes”) that can be introduced into commercial soybean plants to control soybean pathogens

### SUMMARY OF THE INVENTION

Compositions and methods for identifying, selecting and producing Glycine plants (including wild Glycines and Glycine max lines) with enhanced disease resistance are provided. Disease resistant soybean plants and germplasms are also provided.

In some embodiments, methods of identifying a disease resistant soybean plant or germplasm are provided. Such methods may comprise detecting, in the soybean plant or germplasm, a genetic loci or molecular marker (e.g. SNP or a Quantitative Trait Loci (QTL)) associated with enhanced disease resistance, in particular ASR resistance. In some embodiments the genetic loci or molecular marker associates with the presence of a chromosomal interval comprising the nucleotide sequence of SEQ ID NO: 1 or a portion thereof. In particular embodiments, the molecular associated with the chromosomal interval comprising SEQ ID NO: 1 or a portion thereof, is any of the favorable markers listed in Table 1.

In some embodiments, methods of producing a disease resistant soybean plant are provided. Such methods may comprise detecting, in a soybean germplasm, the presence of a genetic loci and/or a genetic marker associated with enhanced pathogen resistance (e.g. ASR) and producing a progeny plant from said soybean germplasm.

In some embodiments, methods of selecting a disease resistant soybean plant or germplasm are provided. Such methods may comprise crossing a first soybean plant or germplasm with a second soybean plant or germplasm, wherein the first soybean plant or germplasm comprises a genetic loci derived from soybean Glycine canescens accession PI446934, or a progeny thereof comprising SEQ ID NO: 1, or a portion thereof, associated with enhanced disease resistance and/or tolerance and selecting a progeny plant or germplasm that possesses the genetic loci.

In some embodiments, methods of introgressing a genetic loci derived from soybean accession number PI446934, or a progeny thereof, associated with enhanced pathogen resistance into a soybean plant or germplasm are provided. Such methods may comprise crossing a first soybean plant or germplasm comprising a chromosomal interval derived from soybean accession number PI446934, or a progeny thereof, associated with enhanced pathogen resistance with a second soybean plant or germplasm that lacks said genetic loci and optionally repeatedly backcrossing progeny plants comprising said genetic allele with the second soybean plant or germplasm to produce a soybean plant (e.g. Glycine max) or germplasm with enhanced pathogen resistance comprising the chromosomal interval derived from soybean accession number PI446934, or a progeny thereof, associated with enhanced pathogen resistance. Progeny comprising the chromosomal interval associated with enhanced pathogen resistance may be identified by detecting, in their genomes, the presence of a marker associated with a chromosomal interval comprising SEQ ID NO: 1 or a portion thereof, wherein the marker is any of the markers listed in Table 1. Chromosomal interval from PI446934, or a progeny thereof, can be introgressed into a Glycine max line through the use of traditional breeding methods.

Soybean plants and/or germplasms identified, produced or selected by the methods of this invention are also provided, as are any progeny and/or seeds derived from a soybean plant or germplasm identified, produced or selected by these methods. In one embodiment, a molecular marker associating with the presence of a chromosomal interval comprising SEQ ID NO: 1 or a portion thereof may be used to identify or select for plant lines resistant to ASR. Further said molecular markers may be located within 20 cM, 10 cM, 5 cM, 4 cM, 3 cM, 2 cM, 1 cM of said chromosomal interval. In another embodiment, said molecular marker may be located within 20 cM, 10 cM, 5 cM, 4 cM, 3 cM, 2 cM, 1 cM of any SNP marker associated with ASR, including any of the favorable markers of Table 1.

Non-naturally occurring soybean seeds, plants and/or germplasms comprising one or genetic loci derived from soybean plant accession number PI446934, or a progeny thereof, that is associated with enhanced pathogen resistance (e.g., enhanced resistance to ASR, Cyst Nematode, Phytophthora, brown stem rot, etc.) are also provided.

A marker associated with enhanced pathogen resistance may comprise, consist essentially of, or consist of a single allele or a combination of alleles at one or more genetic loci derived from PI446934, or a progeny thereof, that associate with enhanced pathogen resistance. In one embodiment the marker is within a chromosomal interval comprising SEQ ID NO: 1, or a portion thereof.

The foregoing and other objects and aspects of the present invention are explained in detail in the drawings and specification set forth below.

### BRIEF DESCRIPTION OF THE SEQUENCE LISTING

SEQ ID NO: 1 is a chromosomal interval derived from Glycine canescens line accession PI 446934 referred to herein as “Scaffold 000454F”. Scaffold 000454F have been mapped to G. canescens chromosome 3.

SEQ ID NOS: 2-226, listed at Table 4, describe the DNA sequence of example assay components, including primers, probes, and target sequences, that can be used to detect and differentiate between favorable and unfavorable alleles associated with a given SNP position within the chromosomal interval of SEQ ID NO: 1.

### BRIEF DESCRIPTION OF THE DRAWINGS

FIG. 1 shows the screening of Glycine canescens (PI446934) for rust resistance against a panel of 16 rust isolates and compares the screening to a rust sensitive accession line.

FIG. 2 illustrates the rust rating scale used to measure plant phenotype.

FIG. 3 shows a depiction of SNPs from Glycine canescens PI446934 that are associated with ASR Resistance.

FIG. 4 shows chromosome mapping for ASR resistance QTL for Glycine canescens PI446934.

FIG. 5 is a marker association map for Glycine canescens (PI446934) where bands indicate regions/intervals of respective chromosomes associated with ASR resistance.

FIG. 6 shows a mapping interval from PI446934 Chromosome 3 associated with ASR resistance.

### DETAILED DESCRIPTION OF THE INVENTION

The presently disclosed subject matter relates at least in part to the identification of a genomic region, such as a chromosomal interval, derived from Glycine canescens accession line PI446934, or a progeny thereof, wherein the chromosomal interval is associated with enhanced pathogen resistance. In embodiments, the chromosomal interval is associated with enhanced resistance to the pathogen Asian soy rust (ASR), which is responsible for causing a disease by the same name. In other embodiments, the chromosomal interval is associated with the pathogen Soybean cyst nematode (SCN), which is responsible for causing a disease by the same name. As such, said chromosomal interval from PI446934, or a progeny thereof, may be introgressed into Glycine max lines via somatic embryo rescue (see for example U.S. Patent Publication 2007/0261139 incorporated by reference herein) or through the use of a Glycine max donor line having introgressed into its genome the genetic region from PI446934, or a progeny thereof, wherein the region comprises SEQ ID NO: 1, or a portion thereof. In some embodiments, the chromosomal interval may be introduced through a cis-genic approach. In still other embodiments, genes (e.g., Resistance genes) from the chromosomal interval comprising SEQ ID NO: 1 may be transgenically expressed or genetically modified (e.g., gene edited via TALEN or CRISPR) in plants to confer disease resistance (e.g. Asian Soy Rust (ASR) resistance).

In embodiments, presence of said chromosomal interval (SEQ ID NO: 1), or portion thereof, in the genome of a plant (e.g., a soybean plant) is associated with increased resistance to pathogens such as ASR, SCN, Stem termination, Stem Canker, Bacterial pustule, root knot nematode, brown stem rot, Frogeye leaf spot, or phytophthora, as compared to a control plant not comprising the chromosomal interval in its genome. In another embodiment, a chromosomal interval, or portion thereof, derived from PI446934, or a progeny thereof, is introduced into a Glycine max line not comprising said chromosomal interval, or portion thereof, wherein said introduction confers in the Glycine max line or it's progeny, increased resistances to disease (e.g. ASR) wherein the said chromosome interval is derived from chromosome 1, 2, 3, 4, 5, 6, 7, 8, 9, 10, 11, 12, 13, 14, 15, 16, 17, 18, 19, or chromosome 20 of Glycine canescens and further wherein said chromosomal interval comprises at least one allele that associates with the trait of increased disease resistance, wherein said allele is any one of the alleles depicted in Table 1. In particular embodiments, said chromosome interval or portion thereof is derived from chromosome 3 of Glycine canescens and comprises at least one allele associated with enhanced disease resistance to ASR as provided in Table 1. In another embodiment, an elite Glycine max plant is provided having introduced into its genome SEQ ID NO: 1, or a portion thereof, and exhibiting increased ASR resistance compared to a control plant not comprising SEQ ID NO: 1 or a portion in its genome.

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

### Definitions

Unless defined otherwise, all technical and scientific terms used herein have the same meaning as commonly understood to one of ordinary skill in the art to which the presently disclosed subject matter belongs.

Although the following terms are believed to be well understood by one of ordinary skill in the art, the following definitions are set forth to facilitate understanding of the presently disclosed subject matter.

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

Nucleotide sequences provided herein are presented in the 5′ to 3′ direction, from left to right and are presented using the standard code for representing nucleotide bases as set forth in 37 CFR §§ 1.821-1.825 and §§ 1.831-1.835 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the terms “a” or “an” or “the” may refer to one or more than one. For example, “a” marker can mean one marker or a plurality of markers.

As used herein, the term “and/or” refers to and encompasses all possible combinations of one or more of the associated listed items, as well as the lack of combinations when interpreted in the alternative (“or”).

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, a “coding sequence” or “CDS” is a nucleic acid sequence that is transcribed into RNA such as mRNA, rRNA, tRNA, snRNA, sense RNA or antisense RNA. In embodiments, the RNA is then translated to produce a protein. In example embodiments, the CDS is derived from a cDNA sequence and includes the sequence of spliced exons of a transcript in DNA notation and does not include any intron or 5′ or 3′-untranslated regions (UTRs). In other example embodiments, the CDS is derived from a genomic DNA sequence and includes the sequence of spliced exons of a transcript in DNA notation as well as one or more introns, and 5′ and/or 3′-untranslated regions (UTRs).

As used herein, a “codon optimized” nucleotide sequence means a nucleotide sequence of a recombinant, transgenic, or synthetic polynucleotide wherein the codons are chosen to reflect the particular codon bias that a host cell or organism may have. This is typically done in such a way as to preserve the amino acid sequence of the polypeptide encoded by the codon optimized nucleotide sequence. In certain embodiments, a nucleotide sequence is codon optimized for the cell (e.g., an animal, plant, fungal or bacterial cell) in which the construct is to be expressed. For example, a construct to be expressed in a plant cell can have all or parts of its sequence codon optimized for expression in a plant. See, for example, U.S. Pat. No. 6,121,014. In embodiments, the polynucleotides provided herein are codon-optimized for expression in a plant cell (e.g., a dicot cell, a monocot cell, a soybean cell) or bacterial cell.

The term “comprise”, “comprises” or “comprising,” when used in this specification, indicates the presence of the stated features, integers, steps, operations, elements, or components, but does not preclude the presence or addition of one or more other features, integers, steps, operations, elements, components, and/or groups thereof.

The term “consists essentially of” (and grammatical variants thereof), as applied to a polynucleotide sequence of this invention, means a polynucleotide sequence that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to express the polynucleotide sequence of at least about 50% or more as compared to the expression level of a polynucleotide sequence consisting of the recited sequence.

As used herein, the term “wild glycine” refers to a perennial Glycine plant, for example any one of G. canescens, G. argyrea, G. clandestine, G. latrobeana, G. albicans, G. aphyonota, G. arenaria, G. curvata, G. cyrtoloba, G. dolichocarpa, G. falcate, G. gracei, G. hirticaulis, G. lactovirens, G. latifolia, G. microphylla, G. montis-douglas, G. peratosa, G. pescadrensis, G. pindanica, G. pullenii, G. rubiginosa, G. stenophita, G. syndetika, or G. tomentella. In particular embodiments, “wild glycine” refers to a Glycine canescens plant. “PI446934” refers to Glycine canescens soybean accession numbers PI446934 respectively.

As used herein, the term “allele” refers to one of two or more different nucleotides or nucleotide sequences that occur at a specific locus. Example alleles that are associated with increased pathogen resistance are disclosed with reference to the markers of Table 1.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” refers to a marker whose presence or absence can be used to predict whether and/or to what extent a plant will display a pathogen resistant or disease resistant phenotype. In one example embodiment, a plant may be selected as having an allele in its genome that is introgressed from a wild glycine and that is associated with enhanced ASR resistance when any of the markers of Table 1 is identified in the plant genome.

A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9: 275 (1993)), an amplified fragment length polymorphism (AFLP) (Vos et al., Nucleic Acids Res. 23: 4407 (1995)), a single nucleotide polymorphism (SNP) (Brookes, Gene 234: 177 (1993)), a sequence-characterized amplified region (SCAR) (Paran and Michelmore, Theor. Appl. Genet. 85: 985 (1993)), a sequence-tagged site (STS) (Onozaki et al., Euphytica 138: 255 (2004)), a single-stranded conformation polymorphism (SSCP) (Orita et al., Proc. Natl. Acad. Sci. USA 86: 2766 (1989)), an inter-simple sequence repeat (ISSR) (Blair et al., Theor. Appl. Genet. 98: 780 (1999)), an inter-retrotransposon amplified polymorphism (IRAP), a retrotransposon-microsatellite amplified polymorphism (REMAP) (Kalendar et al., Theor. Appl. Genet. 98: 704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art (e.g., using PCR).

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval defined by and including” used in reference to particular loci and/or alleles, refers to a chromosomal interval delimited by and encompassing the stated loci, alleles.

As used herein, the terms “cross” or “crossed” or “crossing” refer to the fusion of gametes via pollination to produce progeny (e.g., cells, seeds or plants). The term encompasses both sexual crosses (the cross pollination of one plant by another) and selfing (self-pollination, e.g., when the pollen and ovule are from the same plant).

As used herein, the terms “cultivar” and “variety” refer to a group of similar plants that by structural or genetic features and/or performance can be distinguished from other varieties within the same species.

As used herein, the terms “desired allele”, “favorable allele”, and “allele of interest” are used interchangeably to refer to an allele associated with a desired trait. In some embodiments, a “desired allele” and/or “allele of interest” may be associated with either an increase or a decrease of a feature of a given trait, depending on the nature of the desired phenotype. In some embodiments, a “desired allele” and/or “allele of interest” may be associated with a change in morphology, color, etc. In some embodiments, where the desired allele is associated with enhanced pathogen resistance, presence of the allele in a plant is associated with an increased resistance to a given pathogen relative to a control plant wherein the allele is absent.

As used herein, “disease resistance gene” or “resistance gene” or “R-gene” refers to a nucleic acid having a nucleotide sequence (e.g., DNA sequence) encoding a polypeptide, R-protein, or Resistance protein, that when expressed in a plant cell, is capable of enhancing or improving or increasing a defense or immune response in the plant cell, thereby conferring the plant with increased resistance to one or more plant pathogens. In specific embodiments, the chromosomal intervals of the present invention may comprise the sequence for a disease resistance gene, or R-gene, encoding a resistance protein, or R-protein, that confers enhanced pathogen resistance when expressed in a plant cell.

As used herein, the terms “disease tolerance”, “disease resistance”, “disease tolerant” or “disease resistant” refers to a plant's ability to endure and/or thrive despite being infected with a respective disease. Thus “disease tolerance” or “disease resistance” means a statistically significant decrease or the absence in one or more disease symptoms of a plant caused by a plant pathogen when compared to an appropriate control plant. In some embodiments, an increase in disease tolerance or resistance can be (1) measured by a plant's ability to endure and/or thrive despite being infected with a respective disease; (2) measured by infected disease resistant legume or soybean plants yielding as well as (or nearly as well) as uninfected legume or soybean plants; or (3) measured by a delay or the prevention of proliferation of a pathogen (e.g., fungi), including a delay or the prevention in disease related symptoms. In still other embodiments, a plant or germplasm can be labeled as “disease resistant” if it displays “enhanced or increased pathogen resistance” when compared to a control plant.

As used herein, the terms “enhanced pathogen resistance”, “enhanced disease resistance”, “increased resistance to a pathogen,” or “confers pathogen resistance” refers to an improvement, enhancement, or increase in a plant's ability to endure and/or thrive despite being infected with a pathogen or disease (e.g., Asian soybean rust) as compared to one or more control plants. In embodiments, enhanced disease resistance includes any mechanism (other than whole-plant immunity or resistance) that reduces the expression of symptoms indicative of infection for a given disease or pathogen, such as Asian soybean rust, soybean cyst nematode, Phytophthora, etc. Enhanced disease resistance includes a reduction in the symptoms indicative of infection for a disease such as Asian soybean rust (“ASR”). An enhanced plant pathogen resistance may comprise any statistically significant increase in resistance to the plant pathogen, including, for example, an increase of at least 1%, 5%, 10%, 20%, 30%, 40%, 50%, 60%, 70%, 80%, 90%, 100% or higher. Conferring or enhancing or increasing resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include a statistically significant reduction in the number, size, and/or density of lesions, change in the color of lesions (such as from a tan coloration to a reddish-brown coloration), reduction in number and density of pustule formation, reduction in sporulation, reduction in defoliation, a reduction in yield loss, or any combination thereof. Further, enhanced pathogen resistance can include the prevention or delay of proliferation of a pathogen (e.g., fungus) in the plant.

A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

As used herein, the terms “elite” and “elite line” refer to any line that has resulted from breeding and selection for desirable agronomic performance. An elite line may be substantially homozygous. Numerous elite lines are available and known to those of skill in the art.

As used herein, the term “elite germplasm” refers to any germplasm that is derived from, or is capable of giving rise to, an elite plant.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

A “genetic map” is a description of genetic linkage relationships among loci on one or more chromosomes within a given species, generally depicted in a diagrammatic or tabular form. For each genetic map, distances between loci are measured by the recombination frequencies between them. Recombinations between loci can be detected using a variety of markers. A genetic map is a product of the mapping population, types of markers used, and the polymorphic potential of each marker between different populations. The order and genetic distances between loci can differ from one genetic map to another. In one non-limiting example, distances in a genetic map are indicated in centimorgan (cM) units.

When genetic map loci are represented in cM, position zero corresponds to the first (most distal) marker known at the beginning of the chromosome on both Syngenta's internal consensus genetic map and the Williams 82 v2.0 reference genome map, which is freely available to the public from the soybase(.)org website.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm may refer to seeds, cells (including protoplasts and calli) or tissues from which new plants may be grown, as well as plant parts that can be cultured into a whole plant (e.g., stems, buds, roots, leaves, etc.).

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the term “indel” refers to an insertion or deletion in a pair of nucleotide sequences, wherein a first sequence may be referred to as having an insertion relative to a second sequence or the second sequence may be referred to as having a deletion relative to the first sequence.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, a marker associated with enhanced ASR tolerance may be introgressed from a donor into a recurrent parent that is not Disease resistant. The resulting offspring could then be repeatedly backcrossed and selected until the progeny possess the ASR tolerance allele(s) in the recurrent parent background.

As used herein, the term “linkage” refers to the degree with which one marker locus is associated with another marker locus or some other locus (for example, an ASR tolerance locus). The linkage relationship between a molecular marker and a phenotype may be given as a “probability” or “adjusted probability.” Linkage can be expressed as a desired limit or range. For example, in some embodiments, any marker is linked (genetically and physically) to any other marker when the markers are separated by less than about 50, 40, 30, 25, 20, or 15 map units (or cM).

In some aspects of the present invention, it is advantageous to define a bracketed range of linkage, for example, from about 10 cM and about 20 cM, from about 10 cM and about 30 cM, or from about 10 cM and about 40 cM. The more closely a marker is linked to a second locus, the better an indicator for the second locus that marker becomes. Thus, “closely linked loci” such as a marker locus and a second locus display an inter-locus recombination frequency of about 10%, 9%, 8%, 7%, 6%, 5%, 4%, 3%, or 2% or less. In some embodiments, the relevant loci display a recombination frequency of about 1% or less, e.g., about 0.75%, 0.5%, 0.25% or less. Two loci that are localized to the same chromosome, and at such a distance that recombination between the two loci occurs at a frequency of less than about 10% (e.g., about 9%, 8%, 7%, 6%, 5%, 4%, 3%, 2%, 1%, 0.75%, 0.5%, or 0.25%, or less) may also be said to be “proximal to” each other. Since one cM is the distance between two markers that show a 1% recombination frequency, any marker is closely linked (genetically and physically) to any other marker that is in close proximity, e.g., at or less than about 10 cM distant. Two closely linked markers on the same chromosome may be positioned about 9, 8, 7, 6, 5, 4, 3, 2, 1, 0.75, 0.5 or 0.25 cM or less from each other.

As used herein, the term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and, by definition, are separated by less than 50 cM on the same chromosome). As used herein, linkage can be between two markers, or alternatively between a marker and a phenotype. A marker locus can be “associated with” (linked to) a trait, e.g., Asian Soybean Rust (herein ‘ASR’). The degree of linkage of a molecular marker to a phenotypic trait is measured, e.g., as a statistical probability of co-segregation of that molecular marker with the phenotype.

Linkage disequilibrium is most commonly assessed using the measure r², which is calculated using the formula described by Hill and Robertson, Theor. Appl. Genet. 38:226 (1968). When r²=1, complete linkage disequilibrium exists between the two marker loci, meaning that the markers have not been separated by recombination and have the same allele frequency. Values for r² above ⅓ indicate sufficiently strong linkage disequilibrium to be useful for mapping. Ardlie et al., Nature Reviews Genetics 3:299 (2002). Hence, alleles are in linkage disequilibrium when r² values between pairwise marker loci are greater than or equal to about 0.33, 0.4, 0.5, 0.6, 0.7, 0.8, 0.9, or 1.0.

As used herein, the term “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome where a gene or marker or allele is located. In some embodiments, a locus may encompass one or more nucleotides.

As used herein, the terms “marker” and “genetic marker” are used interchangeably to refer to a nucleotide and/or a nucleotide sequence that has been associated with a phenotype, trait or trait form. In some embodiments, a marker may be associated with an allele or alleles of interest and may be indicative of the presence or absence of the allele or alleles of interest in a cell or organism. A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art. A large number of soybean molecular markers are known in the art, and are published or available from various sources, such as the SoyBase internet resource.

Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), and/or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele,” also described as an “allele of a marker locus,” can refer to one of a plurality of polymorphic nucleotide sequences found at a marker locus in a population that is polymorphic for the marker locus.

“Marker-assisted selection” (MAS) is a process by which phenotypes are selected based on marker genotypes. In some embodiments, marker genotypes are used to identify plants that will be selected for a breeding program or for planting. In some embodiments, marker genotypes are used to identify plants that will not be selected for a breeding program or for planting (i.e., counter-selected plants), allowing them to be removed from the breeding/planting population.

As used herein, the terms “marker locus” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the terms “marker probe” and “probe” refer to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus.

As used herein, the terms “molecular marker” or “genetic marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein are also referred to as hybridization markers when located on an indel region. This is because the insertion region is, by definition, a polymorphism vis-ã-vis a plant without the insertion. Thus, the marker need only indicate whether the indel region is present or absent. Any suitable marker detection technology may be used to identify such a hybridization marker, e.g., SNP technology is used in the examples provided herein.

A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm and crossing a naturally occurring variety of soybean with a non-naturally occurring variety of soybean. In some embodiments, a “non-naturally occurring variety of soybean” may comprise one of more heterologous nucleotide sequences. In some embodiments, a “non-naturally occurring variety of soybean” may comprise one or more non-naturally occurring copies of a naturally occurring nucleotide sequence (i.e., extraneous copies of a gene that naturally occurs in soybean). In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines).

As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, or an electromechanical assay. In some cases, a phenotype or trait is directly controlled by a single gene or genetic locus, i.e., a “single gene trait.” in other cases, a phenotype or trait is the result of several genes. it is noted that, as used herein, the term “disease resistant phenotype” takes into account environmental conditions that might affect the respective disease such that the effect is real and reproducible.

As used herein, the term “plant” may refer to a whole plant, any part thereof, or a cell or tissue culture derived from a plant. Thus, the term “plant” can refer to any of: whole plants, plant components or organs (e.g., roots, stems, leaves, buds, flowers, pods, etc.), plant tissues, seeds and/or plant cells. A plant cell is a cell of a plant, taken from a plant, or derived through culture from a cell taken from a plant. Thus, the term “soybean plant” may refer to a whole soybean plant, one or more parts of a soybean plant (e.g., roots, root tips, stems, leaves, buds, flowers, pods, seeds, cotyledons, etc.), soybean plant cells, soybean plant protoplasts and/or soybean plant calli.

As used herein, the term “polymorphism” refers to a variation in the nucleotide sequence at a locus, where said variation is too common to be due merely to a spontaneous mutation. A polymorphism can be a single nucleotide polymorphism (SNP) or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the term “population” refers to a genetically heterogeneous collection of plants sharing a common genetic derivation.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein, the term “reference sequence” refers to a defined nucleotide sequence used as a basis for nucleotide sequence comparison. The reference sequence for a marker, for example, is obtained by genotyping a number of lines at the locus or loci of interest, aligning the nucleotide sequences in a sequence alignment program, and then obtaining the consensus sequence of the alignment. Hence, a reference sequence identifies the polymorphisms in alleles at a locus. A reference sequence may not be a copy of an actual nucleic acid sequence from any particular organism; however, it is useful for designing primers and probes for actual polymorphisms in the locus or loci.

As used herein, the terms “disease tolerance” and “Disease resistant” refer to a plant's ability to endure and/or thrive despite being infected with a respective disease. When used in reference to germplasm, the terms refer to the ability of a plant that arises from that germplasm to endure and/or thrive despite being infected with a respective disease. In some embodiments, infected Disease resistant soybean plants may yield as well (or nearly as well) as uninfected soybean plants. In general, a plant or germplasm is labeled as “Disease resistant” if it displays “enhanced pathogen resistance.”

As used herein, the terms “enhanced pathogen resistance”, “enhanced disease resistance”, and “conferring or enhancing resistance to a pathogen” refers to an improvement, enhancement, or increase in a plant's ability to endure and/or thrive despite being infected with a pathogen or disease (e.g., Asian soybean rust) as compared to one or more control plants (e.g., one or both of the parents, or a plant lacking the chromosomal interval or marker associated with enhanced pathogen resistance to respective pathogen/disease). The control plants may be fully susceptible to the pathogen or have limited resistance to the pathogen. Enhanced disease resistance includes any mechanism (other than whole-plant immunity or resistance) that reduces the expression of symptoms indicative of infection for a respective disease such as Asian soybean rust, soybean cyst nematode, Phytophthora, etc. Conferring or enhancing of resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include a reduction in the number, size, and/or density of lesions, change in the color of lesions (such as from a tan coloration to a reddish-brown coloration), reduction in number and density of pustule formation, reduction in sporulation, or any combination thereof.

In embodiments, the chromosomal interval of the present invention can be used to enhance pathogen resistance to a fungal pathogen and/or a nematode. As non-limiting examples, the chromosomal interval of the present invention can be used to enhance resistance to: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme.

A “favorable allele” of a marker is a marker allele that segregates with the favorable plant phenotype, therefore providing the benefit of identifying plants that can be selected for a breeding program or planting. In example embodiments, as used herein, a favorable allele of a marker is a marker allele that segregates with the pathogen resistant phenotype of a plant.

An “unfavorable allele” of a marker is a marker allele that segregates with the unfavorable plant phenotype, therefore providing the benefit of identifying plants that can be removed from a breeding program or planting.

Table 1 indicates single nucleotide polymorphisms (SNPs) within SEQ ID NO: 1 that associate with ASR resistance. All alleles for the SNPs identified in Table 1 were determined to be significantly linked with resistance or susceptibility (p<0.05).

TABLE 1
SNP MARKERS WITHIN SEQ ID NO: 1 THAT ARE ASSOCIATED
WITH INCREASED RESISTANCE TO ASR

| | Position | | |
|---|---|---|---|
| | within SEQ ID | Favorable Allele | Unfavorable Allele |
| SNP ID NO. | NO: 1 | (Rust Resistant) | (Rust Susceptible) |
| 1850 | 152261 | G | C |
| 1851 | 152266 | G | A |
| 1852 | 152548 | C | T |
| 1853 | 152555 | C | T |
| 1854 | 152694 | G | A |
| 1855 | 152803 | C | A |
| 1856 | 152839 | T | C |
| 1857 | 152915 | A | C |
| 1858 | 152959 | A | T |
| 1859 | 153137 | A | G |
| 1860 | 154053 | C | T |
| 1861 | 154140 | A | G |
| 1862 | 154285 | C | G |
| 1863 | 154389 | T | C |
| 1864 | 154394 | G | T |
| 1865 | 154520 | C | T |
| 1866 | 154639 | T | C |
| 1867 | 154726 | T | C |
| 1868 | 154855 | T | C |
| 1869 | 154865 | G | A |
| 1870 | 154910 | G | A |
| 1871 | 154940 | T | G |
| 1872 | 155612 | G | T |
| 1873 | 156107 | G | A |
| 1874 | 156134 | T | C |
| 1875 | 156661 | G | A |
| 1876 | 156702 | A | G |
| 1877 | 157081 | A | T |
| 1878 | 157176 | T | C |
| 1879 | 157221 | G | A |
| 1880 | 157351 | G | T |
| 1881 | 157806 | G | T |
| 1882 | 157822 | T | C |
| 1883 | 157976 | A | G |
| 1884 | 158345 | C | T |
| 1885 | 158380 | C | T |
| 1886 | 159057 | G | A |
| 1887 | 159101 | G | C |
| 1888 | 159141 | G | T |
| 1889 | 159157 | T | A |
| 1890 | 159159 | A | G |
| 1891 | 159234 | A | G |
| 1892 | 159247 | G | T |
| 1893 | 160231 | T | G |
| 1894 | 160318 | C | A |
| 1895 | 160413 | T | C |
| 1896 | 160493 | C | T |
| 1897 | 160557 | A | T |
| 1898 | 160568 | G | A |
| 1899 | 160863 | A | C |
| 1900 | 160949 | T | C |
| 1901 | 161299 | T | A |
| 1902 | 161769 | T | A |
| 1903 | 162372 | A | C |
| 1904 | 162401 | G | A |
| 1905 | 162600 | A | T |
| 1906 | 162625 | A | G |
| 1907 | 163062 | T | C |
| 1908 | 163194 | T | A |
| 1909 | 163395 | G | A |
| 1910 | 163469 | T | C |
| 1911 | 163516 | A | G |
| 1912 | 163629 | G | C |
| 1913 | 163681 | G | A |
| 1914 | 163718 | C | G |
| 1915 | 163720 | A | C |
| 1916 | 163781 | C | T |
| 1917 | 163806 | G | A |
| 1918 | 163841 | A | G |
| 1919 | 163856 | T | G |
| 1920 | 163906 | A | T |
| 1921 | 163907 | C | A |
| 1922 | 163978 | C | T |
| 1923 | 164094 | A | G |
| 1924 | 164128 | A | T |
| 1925 | 164249 | G | C |
| 1926 | 164281 | T | G |
| 1927 | 164313 | A | C |
| 1928 | 164340 | T | C |
| 1929 | 164354 | T | G |
| 1930 | 164424 | C | A |
| 1931 | 164511 | C | A |
| 1932 | 164634 | T | C |
| 1933 | 164824 | G | A |
| 1934 | 164891 | C | T |
| 1935 | 164964 | G | A |
| 1936 | 165079 | A | G |
| 1937 | 165144 | G | A |
| 1938 | 165200 | A | G |
| 1939 | 165246 | T | C |
| 1940 | 165365 | C | T |
| 1941 | 165375 | C | T |
| 1942 | 165388 | T | C |
| 1943 | 165414 | T | G |
| 1944 | 165439 | T | C |
| 1945 | 165491 | C | T |
| 1946 | 165495 | A | G |
| 1947 | 165543 | A | T |
| 1948 | 165566 | T | C |
| 1949 | 165628 | A | C |
| 1950 | 165686 | C | T |
| 1951 | 165693 | G | A |
| 1952 | 165702 | G | A |
| 1953 | 165783 | T | C |
| 1954 | 165793 | G | C |
| 1955 | 165810 | T | C |
| 1956 | 165811 | G | A |
| 1957 | 165886 | G | T |
| 1958 | 165897 | T | C |
| 1959 | 165928 | G | T |
| 1960 | 166100 | T | C |
| 1961 | 166101 | T | C |
| 1962 | 166199 | A | C |
| 1963 | 166278 | T | C |
| 1964 | 166346 | A | G |
| 1965 | 166377 | C | T |
| 1966 | 166401 | A | G |
| 1967 | 166437 | G | A |
| 1968 | 166549 | G | A |
| 1969 | 166576 | A | T |
| 1970 | 166577 | A | G |
| 1971 | 166580 | T | C |
| 1972 | 166612 | A | G |
| 1973 | 166681 | A | T |
| 1974 | 166682 | A | T |
| 1975 | 166683 | A | T |
| 1976 | 166737 | A | T |
| 1977 | 166749 | T | A |
| 1978 | 166924 | A | T |
| 1979 | 166964 | T | G |
| 1980 | 167001 | T | A |
| 1981 | 167016 | T | G |
| 1982 | 167170 | T | A |
| 1983 | 167212 | A | G |
| 1984 | 167327 | C | G |
| 1985 | 167563 | C | T |
| 1986 | 167822 | A | T |
| 1987 | 167881 | C | T |
| 1988 | 167954 | — | T |
| 1989 | 167987 | G | A |
| 1990 | 168026 | A | G |
| 1991 | 168295 | C | T |
| 1992 | 168474 | A | G |
| 1993 | 168569 | C | T |
| 1994 | 168595 | T | G |
| 1995 | 168605 | G | A |
| 1996 | 168666 | C | T |
| 1997 | 168722 | T | A |
| 1998 | 168752 | G | A |
| 1999 | 168878 | T | C |
| 2000 | 168908 | C | T |
| 2001 | 168931 | G | A |
| 2002 | 168938 | T | C |
| 2003 | 168946 | G | A |
| 2004 | 168974 | T | A |
| 2005 | 169097 | G | C |
| 2006 | 169284 | G | A |
| 2007 | 169540 | G | A |
| 2008 | 169784 | T | C |
| 2009 | 170568 | T | C |
| 2010 | 170779 | — | TAAT |
| 2011 | 170809 | A | G |
| 2012 | 170859 | T | G |
| 2013 | 170920 | A | C |
| 2014 | 170961 | C | A |
| 2015 | 171107 | C | A |
| 2016 | 171156 | C | T |
| 2017 | 171180 | A | G |
| 2018 | 171194 | G | A |
| 2019 | 171198 | C | T |
| 2020 | 171202 | A | T |
| 2021 | 171229 | T | A |
| 2022 | 171296 | G | T |
| 2023 | 171344 | C | T |
| 2024 | 171378 | G | C |
| 2025 | 171389 | A | T |
| 2026 | 171401 | G | A |
| 2027 | 171474 | C | T |
| 2028 | 171482 | G | T |
| 2029 | 171487 | T | C |
| 2030 | 171563 | G | A |
| 2031 | 171572 | C | T |
| 2032 | 171575 | T | C |
| 2033 | 171616 | A | C |
| 2034 | 171630 | A | G |
| 2035 | 171802 | A | C |
| 2036 | 171827 | C | T |
| 2037 | 171832 | T | G |
| 2038 | 171847 | C | T |
| 2039 | 171879 | A | C |
| 2040 | 171941 | G | A |
| 2041 | 171982 | T | C |
| 2042 | 172001 | T | G |
| 2043 | 172173 | A | C |
| 2044 | 172258 | C | T |
| 2045 | 172371 | — | AAC |
| 2046 | 172391 | G | T |
| 2047 | 172453 | T | C |
| 2048 | 172506 | C | T |
| 2049 | 172557 | G | A |
| 2050 | 172567 | G | A |
| 2051 | 172599 | C | A |
| 2052 | 172635 | G | A |
| 2053 | 172639 | G | A |
| 2054 | 172655 | G | T |
| 2055 | 172672 | C | T |
| 2056 | 172731 | A | T |
| 2057 | 172732 | G | T |
| 2058 | 172742 | C | T |
| 2059 | 172743 | A | G |
| 2060 | 172801 | C | T |
| 2061 | 172822 | A | G |
| 2062 | 172847 | G | A |
| 2063 | 172894 | C | A |
| 2064 | 172920 | C | T |
| 2065 | 172950 | A | G |
| 2066 | 173012 | A | G |
| 2067 | 173068 | A | G |
| 2068 | 173075 | A | G |
| 2069 | 173076 | G | T |
| 2070 | 173224 | A | T |
| 2071 | 173331 | C | T |
| 2072 | 173335 | A | G |
| 2073 | 173394 | C | T |
| 2074 | 173428 | T | C |
| 2075 | 173432 | — | T |
| 2076 | 173545 | A | T |
| 2077 | 173693 | T | C |
| 2078 | 173723 | T | G |
| 2079 | 173766 | C | T |
| 2080 | 173945 | T | G |
| 2081 | 174061 | A | T |
| 2082 | 174262 | C | A |
| 2083 | 174534 | C | T |
| 2084 | 174538 | A | C |
| 2085 | 174618 | G | A |
| 2086 | 174626 | G | T |
| 2087 | 174665 | T | A |
| 2088 | 174838 | A | C |
| 2089 | 174907 | C | T |
| 2090 | 179196 | T | C |
| 2091 | 179606 | T | C |
| 2092 | 179703 | A | C |
| 2093 | 180763 | C | T |
| 2094 | 180879 | A | C |
| 2095 | 180934 | A | G |
| 2096 | 180944 | A | G |
| 2097 | 181068 | A | G |
| 2098 | 181193 | T | C |
| 2099 | 181402 | T | G |
| 2100 | 181628 | T | C |
| 2101 | 181680 | C | T |
| 2102 | 181733 | A | G |
| 2103 | 181755 | T | G |
| 2104 | 182699 | T | C |
| 2105 | 182702 | T | C |
| 2106 | 182714 | A | G |
| 2107 | 182811 | A | G |
| 2108 | 183078 | T | C |
| 2109 | 183139 | C | T |
| 2110 | 183151 | C | T |
| 2111 | 183446 | T | A |
| 2112 | 183489 | T | C |
| 2113 | 183698 | T | C |
| 2114 | 183713 | G | T |
| 2115 | 183721 | A | G |
| 2116 | 184257 | A | T |
| 2117 | 184302 | A | G |
| 2118 | 184573 | C | A |
| 2119 | 184895 | T | C |
| 2120 | 185013 | C | T |
| 2121 | 185139 | C | T |
| 2122 | 185446 | C | T |
| 2123 | 185525 | A | G |
| 2124 | 185565 | G | C |
| 2125 | 185580 | A | G |
| 2126 | 185617 | A | C |
| 2127 | 185635 | A | T |
| 2128 | 185809 | T | C |
| 2129 | 185969 | T | C |
| 2130 | 186041 | T | C |
| 2131 | 186063 | T | C |
| 2132 | 186064 | A | G |
| 2133 | 186069 | C | G |
| 2134 | 186100 | T | C |
| 2135 | 186173 | T | C |
| 2136 | 186633 | T | C |
| 2137 | 186757 | G | A |
| 2138 | 186847 | A | G |
| 2139 | 186980 | A | G |
| 2140 | 187136 | T | C |
| 2141 | 187157 | A | G |
| 2142 | 187170 | A | G |
| 2143 | 187185 | A | C |
| 2144 | 187312 | T | C |
| 2145 | 187489 | T | C |
| 2146 | 187997 | A | G |
| 2147 | 188101 | C | T |
| 2148 | 188247 | A | G |
| 2149 | 188300 | C | T |
| 2150 | 188422 | A | T |
| 2151 | 189186 | T | C |
| 2152 | 189223 | T | A |
| 2153 | 189591 | T | C |
| 2154 | 190003 | T | C |
| 2155 | 190080 | T | C |
| 2156 | 190127 | A | G |
| 2157 | 190689 | C | T |
| 2158 | 190737 | G | A |
| 2159 | 190819 | T | C |
| 2160 | 190827 | A | C |
| 2161 | 190840 | A | G |
| 2162 | 190851 | C | A |
| 2163 | 190882 | A | C |
| 2164 | 190932 | G | A |
| 2165 | 190947 | G | A |
| 2166 | 191037 | G | A |
| 2167 | 191135 | C | T |
| 2168 | 191195 | T | C |
| 2169 | 191231 | A | G |
| 2170 | 191276 | A | G |
| 2171 | 191312 | G | C |
| 2172 | 191371 | T | C |
| 2173 | 191439 | T | C |
| 2174 | 191474 | T | G |
| 2175 | 191506 | T | C |
| 2176 | 191595 | A | T |
| 2177 | 191631 | A | G |
| 2178 | 191648 | A | C |
| 2179 | 191762 | T | C |
| 2180 | 191853 | T | C |
| 2181 | 191945 | G | A |
| 2182 | 192012 | A | G |
| 2183 | 192037 | T | A |
| 2184 | 192076 | C | T |
| 2185 | 192155 | C | A |
| 2186 | 192211 | C | A |
| 2187 | 192212 | G | A |
| 2188 | 192218 | T | C |
| 2189 | 192349 | T | G |
| 2190 | 192453 | A | C |
| 2191 | 192455 | A | G |
| 2192 | 192537 | C | T |
| 2193 | 192579 | A | C |
| 2194 | 192633 | C | T |
| 2195 | 192664 | G | A |
| 2196 | 192697 | G | A |
| 2197 | 192702 | C | G |
| 2198 | 192813 | G | C |
| 2199 | 192870 | A | G |
| 2200 | 192884 | A | C |
| 2201 | 192951 | T | C |
| 2202 | 192980 | T | G |
| 2203 | 193281 | G | A |
| 2204 | 193327 | T | C |
| 2205 | 193345 | A | G |
| 2206 | 193450 | T | C |
| 2207 | 193451 | G | A |
| 2208 | 193675 | T | C |
| 2209 | 193736 | A | C |
| 2210 | 193779 | T | C |
| 2211 | 193806 | C | T |
| 2212 | 193897 | C | T |
| 2213 | 193901 | A | G |
| 2214 | 193956 | G | A |
| 2215 | 194015 | T | G |
| 2216 | 194041 | T | G |
| 2217 | 194078 | T | C |
| 2218 | 194105 | A | G |
| 2219 | 194185 | A | G |
| 2220 | 194191 | T | C |
| 2221 | 194218 | G | A |
| 2222 | 194342 | T | G |
| 2223 | 194381 | C | T |
| 2224 | 194506 | C | T |
| 2225 | 194534 | C | T |
| 2226 | 194629 | G | A |
| 2227 | 194640 | A | G |
| 2228 | 194645 | A | C |
| 2229 | 194676 | G | A |
| 2230 | 194683 | T | C |
| 2231 | 194734 | A | G |
| 2232 | 194831 | T | C |
| 2233 | 194986 | T | C |
| 2234 | 194993 | C | A |
| 2235 | 195046 | — | C |
| 2236 | 195081 | A | C |
| 2237 | 195100 | T | C |
| 2238 | 195115 | T | C |
| 2239 | 195181 | A | G |
| 2240 | 195192 | A | G |
| 2241 | 195225 | A | G |
| 2242 | 195240 | T | C |
| 2243 | 195279 | G | A |
| 2244 | 195332 | T | C |
| 2245 | 195353 | T | C |
| 2246 | 195361 | T | C |
| 2247 | 195421 | C | T |
| 2248 | 195494 | T | C |
| 2249 | 195529 | T | C |
| 2250 | 195602 | T | C |
| 2251 | 195690 | C | T |
| 2252 | 195728 | T | C |
| 2253 | 195738 | G | A |
| 2254 | 195801 | C | T |
| 2255 | 195900 | T | A |
| 2256 | 195922 | C | T |
| 2257 | 195924 | C | T |
| 2258 | 196258 | A | G |
| 2259 | 196335 | T | A |
| 2260 | 196383 | T | C |
| 2261 | 196438 | G | A |
| 2262 | 196605 | G | A |
| 2263 | 196621 | C | T |
| 2264 | 196649 | T | C |
| 2265 | 196786 | A | G |
| 2266 | 196857 | C | T |
| 2267 | 196883 | G | T |
| 2268 | 196894 | T | C |
| 2269 | 196920 | T | C |
| 2270 | 196932 | A | G |
| 2271 | 196936 | A | C |
| 2272 | 196970 | A | G |
| 2273 | 196971 | T | C |
| 2274 | 196972 | C | T |
| 2275 | 197049 | C | T |
| 2276 | 197059 | T | G |
| 2277 | 197103 | A | G |
| 2278 | 197108 | G | A |
| 2279 | 197144 | T | C |
| 2280 | 197238 | C | G |
| 2281 | 197246 | T | C |
| 2282 | 197283 | G | C |
| 2283 | 197285 | A | T |
| 2284 | 197316 | C | T |
| 2285 | 197352 | C | T |
| 2286 | 197384 | A | G |
| 2287 | 197390 | A | G |
| 2288 | 197409 | G | A |
| 2289 | 197509 | A | T |
| 2290 | 197539 | G | A |
| 2291 | 197544 | T | C |
| 2292 | 197594 | A | G |
| 2293 | 197617 | G | A |
| 2294 | 197650 | G | T |
| 2295 | 197672 | A | G |
| 2296 | 197676 | C | G |
| 2297 | 197860 | T | C |
| 2298 | 197959 | A | G |
| 2299 | 197964 | A | C |
| 2300 | 197987 | T | C |
| 2301 | 198040 | T | C |
| 2302 | 198081 | A | T |
| 2303 | 198156 | C | G |
| 2304 | 198220 | A | G |
| 2305 | 198249 | A | G |
| 2306 | 198397 | A | G |
| 2307 | 198694 | G | A |
| 2308 | 198775 | T | G |
| 2309 | 198778 | A | T |
| 2310 | 198823 | C | T |
| 2311 | 198892 | G | A |
| 2312 | 198900 | T | G |
| 2313 | 198963 | A | C |
| 2314 | 198989 | A | C |
| 2315 | 199039 | T | C |
| 2316 | 199055 | T | C |
| 2317 | 199155 | A | G |
| 2318 | 199166 | A | G |
| 2319 | 199237 | A | G |
| 2320 | 199561 | T | C |
| 2321 | 199614 | A | G |
| 2322 | 199647 | C | T |
| 2323 | 199695 | A | G |
| 2324 | 199898 | C | T |
| 2325 | 199974 | C | A |
| 2326 | 200033 | G | T |
| 2327 | 200076 | A | G |
| 2328 | 200108 | G | A |
| 2329 | 200137 | G | A |
| 2330 | 200403 | A | G |
| 2331 | 200633 | G | A |
| 2332 | 200884 | G | T |
| 2333 | 201025 | T | A |
| 2334 | 201677 | A | G |
| 2335 | 201709 | T | G |
| 2336 | 201806 | T | C |
| 2337 | 201981 | G | A |
| 2338 | 203223 | T | A |
| 2339 | 203243 | C | T |
| 2340 | 203430 | A | G |
| 2341 | 203539 | A | C |
| 2342 | 203552 | A | G |
| 2343 | 203592 | G | A |
| 2344 | 203656 | T | A |
| 2345 | 203793 | T | C |
| 2346 | 203841 | A | T |
| 2347 | 203876 | G | A |
| 2348 | 203943 | C | A |
| 2349 | 204053 | A | G |
| 2350 | 204165 | A | T |
| 2351 | 204284 | T | C |
| 2352 | 204429 | C | A |
| 2353 | 204451 | C | G |
| 2354 | 204610 | — | A |
| 2355 | 204670 | A | T |
| 2356 | 204725 | G | T |
| 2357 | 204992 | C | T |
| 2358 | 205121 | A | C |
| 2359 | 205198 | G | A |
| 2360 | 205210 | C | A |
| 2361 | 205236 | T | A |
| 2362 | 205257 | T | C |
| 2363 | 205291 | A | G |
| 2364 | 205393 | A | T |
| 2365 | 205546 | — | T |
| 2366 | 205596 | G | T |
| 2367 | 205686 | A | G |
| 2368 | 205705 | A | G |
| 2369 | 205710 | T | C |
| 2370 | 205978 | A | T |
| 2371 | 206187 | G | A |
| 2372 | 206501 | A | C |
| 2373 | 206523 | G | A |
| 2374 | 207402 | C | T |
| 2375 | 207423 | C | T |
| 2376 | 207425 | G | A |
| 2377 | 207690 | A | G |
| 2378 | 207718 | C | T |
| 2379 | 207789 | C | T |
| 2380 | 207864 | T | A |
| 2381 | 208048 | G | A |
| 2382 | 208053 | C | G |
| 2383 | 208131 | A | C |
| 2384 | 208168 | — | C |
| 2385 | 208214 | C | T |
| 2386 | 208254 | A | G |
| 2387 | 208257 | A | G |
| 2388 | 208261 | G | A |
| 2389 | 208600 | G | A |
| 2390 | 208622 | C | T |
| 2391 | 209018 | G | A |
| 2392 | 209034 | A | G |
| 2393 | 209040 | A | G |
| 2394 | 209046 | G | A |
| 2395 | 209067 | G | A |
| 2396 | 209088 | C | T |
| 2397 | 209129 | G | T |
| 2398 | 209222 | G | A |
| 2399 | 209256 | C | T |
| 2400 | 209303 | — | A |
| 2401 | 209362 | C | G |
| 2402 | 209384 | C | G |
| 2403 | 209428 | G | A |
| 2404 | 209516 | G | C |
| 2405 | 209543 | C | T |
| 2406 | 209544 | A | G |
| 2407 | 209603 | G | A |
| 2408 | 209604 | A | T |
| 2409 | 209613 | T | G |
| 2410 | 209614 | C | T |
| 2411 | 209631 | T | C |
| 2412 | 209712 | T | C |
| 2413 | 209745 | G | A |
| 2414 | 209789 | C | T |
| 2415 | 210043 | G | A |
| 2416 | 210213 | C | T |
| 2417 | 210248 | G | A |
| 2418 | 210271 | C | T |
| 2419 | 210435 | T | C |
| 2420 | 210437 | A | G |
| 2421 | 210511 | C | T |
| 2422 | 210526 | T | A |
| 2423 | 210561 | A | G |
| 2424 | 210579 | A | G |
| 2425 | 210690 | A | C |
| 2426 | 210726 | A | C |
| 2427 | 210803 | C | T |
| 2428 | 210820 | G | A |
| 2429 | 210835 | A | G |
| 2430 | 210876 | T | C |
| 2431 | 210885 | A | C |
| 2432 | 210911 | T | C |
| 2433 | 211174 | A | C |
| 2434 | 211202 | G | A |
| 2435 | 211222 | C | T |
| 2436 | 211318 | C | T |
| 2437 | 211325 | A | T |
| 2438 | 211340 | T | A |
| 2439 | 211431 | C | A |
| 2440 | 211490 | A | G |
| 2441 | 211531 | C | G |
| 2442 | 211585 | A | T |
| 2443 | 211630 | C | G |
| 2444 | 211634 | T | C |
| 2445 | 211680 | T | C |
| 2446 | 211883 | C | T |
| 2447 | 211933 | A | G |
| 2448 | 212099 | T | A |
| 2449 | 212108 | C | T |
| 2450 | 212115 | T | C |
| 2451 | 212135 | A | G |
| 2452 | 212258 | T | C |
| 2453 | 212324 | A | T |
| 2454 | 212377 | A | G |
| 2455 | 212393 | T | G |
| 2456 | 212439 | A | C |
| 2457 | 212454 | C | T |
| 2458 | 212521 | T | G |
| 2459 | 212608 | C | T |
| 2460 | 212628 | C | T |
| 2461 | 212669 | A | G |
| 2462 | 212713 | A | G |
| 2463 | 212782 | C | A |
| 2464 | 212826 | C | T |
| 2465 | 212927 | A | G |
| 2466 | 213032 | T | C |
| 2467 | 213068 | A | G |
| 2468 | 213108 | G | A |
| 2469 | 213244 | C | T |
| 2470 | 213302 | A | G |
| 2471 | 213373 | T | G |
| 2472 | 213422 | A | C |
| 2473 | 213547 | T | C |
| 2474 | 213580 | A | G |
| 2475 | 213647 | T | C |
| 2476 | 213757 | C | T |
| 2477 | 213804 | C | A |
| 2478 | 213819 | G | T |
| 2479 | 213876 | C | T |
| 2480 | 213948 | A | T |
| 2481 | 214003 | G | A |
| 2482 | 214004 | A | G |
| 2483 | 214033 | C | A |
| 2484 | 214041 | C | T |
| 2485 | 214115 | A | G |
| 2486 | 214267 | G | A |
| 2487 | 214303 | C | T |
| 2488 | 214354 | C | T |
| 2489 | 214425 | C | T |
| 2490 | 214474 | T | C |
| 2491 | 214549 | C | T |
| 2492 | 214583 | C | T |
| 2493 | 214599 | G | T |
| 2494 | 214642 | A | T |
| 2495 | 214660 | G | A |
| 2496 | 214707 | A | G |
| 2497 | 214745 | G | A |
| 2498 | 214762 | G | A |
| 2499 | 214840 | A | C |
| 2500 | 214848 | A | G |
| 2501 | 214852 | T | C |
| 2502 | 214854 | G | A |
| 2503 | 214927 | C | T |
| 2504 | 215000 | T | C |
| 2505 | 215001 | G | A |
| 2506 | 215044 | T | C |
| 2507 | 215049 | T | A |
| 2508 | 215076 | A | G |
| 2509 | 215101 | A | G |
| 2510 | 215104 | T | C |
| 2511 | 215139 | T | G |
| 2512 | 215335 | C | T |
| 2513 | 215540 | T | A |
| 2514 | 215548 | G | A |
| 2515 | 215549 | T | C |
| 2516 | 215616 | T | C |
| 2517 | 215926 | C | T |
| 2518 | 216047 | — | A |
| 2519 | 216092 | T | C |
| 2520 | 216170 | G | C |
| 2521 | 216212 | C | A |
| 2522 | 216230 | C | T |
| 2523 | 216263 | T | C |
| 2524 | 216361 | T | G |
| 2525 | 216412 | C | T |
| 2526 | 216413 | G | A |
| 2527 | 216437 | G | C |
| 2528 | 216469 | C | T |
| 2529 | 216480 | A | G |
| 2530 | 216499 | T | G |
| 2531 | 216529 | C | G |
| 2532 | 216647 | C | T |
| 2533 | 216708 | T | A |
| 2534 | 216720 | A | G |
| 2535 | 216765 | A | G |
| 2536 | 216781 | G | A |
| 2537 | 216803 | T | G |
| 2538 | 216834 | A | G |
| 2539 | 216838 | G | A |
| 2540 | 216869 | G | T |
| 2541 | 216993 | C | T |
| 2542 | 217009 | A | C |
| 2543 | 217044 | A | G |
| 2544 | 217133 | T | G |
| 2545 | 217163 | C | A |
| 2546 | 217169 | C | T |
| 2547 | 217173 | C | T |
| 2548 | 217175 | A | G |
| 2549 | 217309 | G | A |
| 2550 | 217477 | C | A |
| 2551 | 217658 | G | A |
| 2552 | 217812 | A | C |
| 2553 | 217915 | G | A |
| 2554 | 217954 | C | T |
| 2555 | 217955 | A | G |
| 2556 | 217986 | T | C |
| 2557 | 218249 | G | A |
| 2558 | 218286 | G | A |
| 2559 | 218409 | A | C |
| 2560 | 218504 | G | T |
| 2561 | 218532 | A | T |
| 2562 | 218533 | A | G |
| 2563 | 218536 | A | T |
| 2564 | 218606 | A | G |
| 2565 | 218641 | T | C |
| 2566 | 218671 | A | C |
| 2567 | 218807 | C | T |
| 2568 | 218935 | G | A |
| 2569 | 219430 | A | G |
| 2570 | 219500 | T | C |
| 2571 | 219578 | T | A |
| 2572 | 219627 | A | G |
| 2573 | 219634 | A | G |
| 2574 | 219675 | T | G |
| 2575 | 219794 | A | C |
| 2576 | 219891 | A | G |
| 2577 | 219963 | C | T |
| 2578 | 220079 | T | G |
| 2579 | 220191 | G | A |
| 2580 | 220217 | A | G |
| 2581 | 220258 | T | C |
| 2582 | 220286 | C | A |
| 2583 | 220376 | T | C |
| 2584 | 220383 | A | G |
| 2585 | 220403 | C | T |
| 2586 | 220446 | — | AA |
| 2587 | 220447 | G | A |
| 2588 | 220607 | A | G |
| 2589 | 220610 | G | A |
| 2590 | 220663 | C | T |
| 2591 | 220712 | A | T |
| 2592 | 220759 | A | T |
| 2593 | 220778 | C | A |
| 2594 | 220780 | T | C |
| 2595 | 220800 | G | A |
| 2596 | 220815 | A | G |
| 2597 | 220854 | T | G |
| 2598 | 220860 | G | A |
| 2599 | 220873 | G | A |
| 2600 | 220888 | T | C |
| 2601 | 220927 | T | C |
| 2602 | 220936 | A | G |
| 2603 | 221034 | A | G |
| 2604 | 221292 | A | G |
| 2605 | 221296 | G | C |
| 2606 | 221352 | C | T |
| 2607 | 221407 | C | T |
| 2608 | 221438 | T | C |
| 2609 | 221555 | G | A |
| 2610 | 221586 | C | A |
| 2611 | 221590 | A | G |
| 2612 | 221598 | — | T |
| 2613 | 221644 | G | A |
| 2614 | 221666 | T | G |
| 2615 | 221691 | G | A |
| 2616 | 221849 | A | G |
| 2617 | 221879 | C | T |
| 2618 | 221897 | — | G |
| 2619 | 221921 | A | G |
| 2620 | 221945 | T | A |
| 2621 | 221956 | A | G |
| 2622 | 221957 | C | T |
| 2623 | 221992 | C | T |
| 2624 | 222014 | C | T |
| 2625 | 222334 | A | G |
| 2626 | 222375 | G | A |
| 2627 | 222524 | G | A |
| 2628 | 222603 | C | T |
| 2629 | 222612 | G | A |
| 2630 | 222660 | G | A |
| 2631 | 222828 | T | C |
| 2632 | 222838 | A | G |
| 2633 | 222964 | C | T |
| 2634 | 222971 | A | G |
| 2635 | 222987 | G | A |
| 2636 | 223113 | G | A |
| 2637 | 223181 | A | C |
| 2638 | 223207 | C | T |
| 2639 | 223248 | G | A |
| 2640 | 223262 | A | G |
| 2641 | 223277 | G | T |
| 2642 | 223376 | C | T |
| 2643 | 223398 | A | G |
| 2644 | 223590 | C | T |
| 2645 | 223742 | A | T |
| 2646 | 223786 | C | T |
| 2647 | 223840 | G | A |
| 2648 | 224330 | G | A |
| 2649 | 224395 | G | T |
| 2650 | 224458 | G | T |
| 2651 | 224465 | A | G |
| 2652 | 224516 | G | A |
| 2653 | 224577 | A | T |
| 2654 | 224612 | C | T |
| 2655 | 224730 | A | G |
| 2656 | 225053 | G | A |
| 2657 | 225183 | T | C |
| 2658 | 225207 | T | C |
| 2659 | 225259 | G | A |
| 2660 | 225342 | T | A |
| 2661 | 225440 | T | A |
| 2662 | 225441 | A | T |
| 2663 | 225450 | G | T |
| 2664 | 225487 | C | T |
| 2665 | 225527 | C | T |
| 2666 | 225596 | T | G |
| 2667 | 225612 | A | C |
| 2668 | 225729 | T | C |
| 2669 | 225795 | C | T |
| 2670 | 225883 | T | C |
| 2671 | 226003 | C | A |
| 2672 | 226045 | T | C |
| 2673 | 226053 | T | A |
| 2674 | 226177 | T | A |
| 2675 | 226178 | T | A |
| 2676 | 226187 | A | G |
| 2677 | 226238 | T | C |
| 2678 | 226544 | G | A |
| 2679 | 226632 | G | A |
| 2680 | 226653 | C | T |
| 2681 | 226705 | T | C |
| 2682 | 226903 | T | C |
| 2683 | 226909 | A | C |
| 2684 | 226931 | G | A |
| 2685 | 226943 | C | A |
| 2686 | 227021 | T | G |
| 2687 | 227161 | A | G |
| 2688 | 227299 | G | A |
| 2689 | 227338 | T | C |
| 2690 | 227454 | C | T |
| 2691 | 227668 | A | G |
| 2692 | 227739 | C | A |
| 2693 | 227793 | A | G |
| 2694 | 227885 | G | A |
| 2695 | 228027 | C | T |
| 2696 | 228059 | G | A |
| 2697 | 228064 | T | C |
| 2698 | 228118 | G | A |
| 2699 | 228150 | G | T |
| 2700 | 228273 | A | T |
| 2701 | 228386 | G | A |
| 2702 | 228579 | G | A |
| 2703 | 228633 | C | A |
| 2704 | 228687 | T | C |
| 2705 | 228712 | T | C |
| 2706 | 228741 | C | A |
| 2707 | 228847 | C | T |
| 2708 | 228854 | C | T |
| 2709 | 228861 | G | A |
| 2710 | 228998 | G | A |
| 2711 | 229010 | A | G |
| 2712 | 229105 | G | A |
| 2713 | 229119 | T | G |
| 2714 | 229129 | G | A |
| 2715 | 229130 | C | T |
| 2716 | 229133 | G | A |
| 2717 | 229150 | T | C |
| 2718 | 229181 | A | G |
| 2719 | 229226 | C | T |
| 2720 | 229302 | T | A |
| 2721 | 229338 | C | T |
| 2722 | 229351 | T | C |
| 2723 | 229375 | T | C |
| 2724 | 229404 | T | C |
| 2725 | 229451 | A | T |
| 2726 | 229461 | A | G |
| 2727 | 229587 | C | A |
| 2728 | 229609 | T | C |
| 2729 | 229618 | C | G |
| 2730 | 229666 | C | T |
| 2731 | 230603 | G | A |
| 2732 | 230885 | T | A |
| 2733 | 231349 | A | G |
| 2734 | 231858 | T | A |
| 2735 | 231864 | T | A |
| 2736 | 231866 | A | G |
| 2737 | 231879 | T | C |
| 2738 | 232072 | A | C |
| 2739 | 232087 | T | A |
| 2740 | 232173 | G | C |
| 2741 | 232263 | T | A |
| 2742 | 232290 | G | A |
| 2743 | 232293 | T | C |
| 2744 | 232315 | C | T |
| 2745 | 232440 | C | G |
| 2746 | 232471 | A | G |
| 2747 | 232554 | T | C |
| 2748 | 232576 | — | T |
| 2749 | 232705 | C | T |
| 2750 | 232714 | A | G |
| 2751 | 232745 | A | G |
| 2752 | 232990 | T | C |
| 2753 | 233013 | A | G |
| 2754 | 233140 | T | C |
| 2755 | 233186 | G | A |
| 2756 | 233253 | C | G |
| 2757 | 233254 | A | T |
| 2758 | 233456 | A | G |
| 2759 | 233468 | T | A |
| 2760 | 233471 | T | C |
| 2761 | 233493 | T | G |
| 2762 | 233526 | C | T |
| 2763 | 233615 | A | G |
| 2764 | 234282 | A | G |
| 2765 | 234480 | A | T |
| 2766 | 234481 | A | T |
| 2767 | 234484 | A | G |
| 2768 | 234505 | G | A |
| 2769 | 234519 | T | C |
| 2770 | 234598 | A | T |
| 2771 | 234641 | A | T |
| 2772 | 234825 | T | G |
| 2773 | 234862 | A | T |
| 2774 | 234872 | T | G |
| 2775 | 234879 | G | C |
| 2776 | 234880 | G | C |
| 2777 | 234959 | G | T |
| 2778 | 235123 | T | C |
| 2779 | 235162 | G | A |
| 2780 | 235170 | A | C |
| 2781 | 235202 | A | T |
| 2782 | 235242 | T | A |
| 2783 | 235244 | T | A |
| 2784 | 235313 | A | C |
| 2785 | 235372 | T | A |
| 2786 | 235868 | G | A |
| 2787 | 235898 | G | A |
| 2788 | 235904 | T | A |
| 2789 | 236043 | C | T |
| 2790 | 236083 | G | C |
| 2791 | 236084 | A | G |
| 2792 | 236089 | C | T |
| 2793 | 236431 | T | C |
| 2794 | 236447 | T | G |
| 2795 | 243825 | A | C |
| 2796 | 243844 | A | G |
| 2797 | 246201 | A | G |
| 2798 | 246217 | T | C |
| 2799 | 246222 | T | G |
| 2800 | 246226 | C | A |
| 2801 | 246236 | T | G |
| 2802 | 246698 | T | C |
| 2803 | 246713 | G | A |
| 2804 | 246716 | C | T |
| 2805 | 246889 | G | T |
| 2806 | 246925 | A | G |
| 2807 | 246990 | T | A |
| 2808 | 246992 | T | A |
| 2809 | 247632 | T | C |
| 2810 | 247678 | T | C |
| 2811 | 247731 | G | A |
| 2812 | 247743 | C | T |
| 2813 | 247763 | A | G |
| 2814 | 247983 | C | G |
| 2815 | 248245 | A | G |
| 2816 | 248811 | C | T |
| 2817 | 248824 | T | C |
| 2818 | 249271 | A | G |
| 2819 | 249284 | G | A |
| 2820 | 252872 | A | C |
| 2821 | 256931 | C | T |
| 2822 | 256934 | T | C |
| 2823 | 257038 | C | T |
| 2824 | 257089 | G | T |
| 2825 | 257150 | T | C |
| 2826 | 257346 | C | T |
| 2827 | 257364 | A | T |
| 2828 | 257366 | C | T |
| 2829 | 257461 | T | A |
| 2830 | 257465 | G | T |
| 2831 | 257484 | T | G |
| 2832 | 257504 | T | A |
| 2833 | 257532 | G | T |
| 2834 | 257588 | G | A |
| 2835 | 257644 | A | T |
| 2836 | 257648 | G | A |
| 2837 | 257657 | A | G |
| 2838 | 257671 | A | G |
| 2839 | 257699 | T | A |
| 2840 | 257719 | G | C |
| 2841 | 257733 | C | T |
| 2842 | 257811 | G | A |
| 2843 | 257843 | C | A |
| 2844 | 257853 | C | T |
| 2845 | 257918 | G | T |
| 2846 | 257920 | A | C |
| 2847 | 257929 | C | T |
| 2848 | 257930 | A | C |
| 2849 | 257983 | T | C |
| 2850 | 258055 | G | A |
| 2851 | 258094 | G | A |
| 2852 | 258097 | G | T |
| 2853 | 258102 | T | C |
| 2854 | 258106 | G | A |
| 2855 | 258132 | C | T |
| 2856 | 258165 | T | C |
| 2857 | 258200 | T | C |
| 2858 | 258217 | A | G |
| 2859 | 258218 | A | T |
| 2860 | 258231 | T | C |
| 2861 | 258245 | C | T |
| 2862 | 258338 | G | T |
| 2863 | 258388 | A | C |
| 2864 | 258389 | T | C |
| 2865 | 258392 | C | G |
| 2866 | 258395 | C | T |
| 2867 | 258489 | A | T |
| 2868 | 258536 | A | G |
| 2869 | 258583 | T | C |
| 2870 | 258584 | A | G |
| 2871 | 258626 | A | G |
| 2872 | 258640 | G | A |
| 2873 | 258687 | A | G |
| 2874 | 258696 | T | C |
| 2875 | 258700 | T | C |
| 2876 | 258726 | A | G |
| 2877 | 258734 | C | T |
| 2878 | 258800 | T | G |
| 2879 | 258804 | T | C |
| 2880 | 258805 | G | A |
| 2881 | 258810 | A | T |
| 2882 | 258830 | G | C |
| 2883 | 258846 | T | C |
| 2884 | 258851 | T | G |
| 2885 | 258873 | T | C |
| 2886 | 258896 | A | G |
| 2887 | 258957 | C | T |
| 2888 | 258963 | C | G |
| 2889 | 258965 | A | G |
| 2890 | 258991 | T | C |
| 2891 | 259007 | C | T |
| 2892 | 259024 | C | G |
| 2893 | 259069 | C | T |
| 2894 | 259095 | C | A |
| 2895 | 259216 | C | T |
| 2896 | 259233 | G | A |
| 2897 | 259235 | T | A |
| 2898 | 259276 | T | C |
| 2899 | 259308 | C | A |
| 2900 | 259427 | A | G |
| 2901 | 259473 | T | C |
| 2902 | 259474 | A | G |
| 2903 | 259484 | A | C |
| 2904 | 259486 | A | G |
| 2905 | 259490 | T | C |
| 2906 | 259504 | A | T |
| 2907 | 259575 | G | A |
| 2908 | 259605 | T | C |
| 2909 | 259690 | C | T |
| 2910 | 259726 | C | T |
| 2911 | 259736 | C | A |
| 2912 | 259760 | C | T |
| 2913 | 259924 | G | T |
| 2914 | 260002 | G | C |
| 2915 | 260140 | G | C |
| 2916 | 260225 | G | A |
| 2917 | 260294 | A | G |
| 2918 | 260341 | T | C |
| 2919 | 260394 | A | G |
| 2920 | 260546 | A | G |
| 2921 | 260556 | C | T |
| 2922 | 260572 | T | G |
| 2923 | 260599 | A | G |
| 2924 | 260611 | G | C |
| 2925 | 260612 | A | C |
| 2926 | 260616 | G | A |
| 2927 | 260677 | T | A |
| 2928 | 260713 | G | C |
| 2929 | 260715 | T | G |
| 2930 | 260854 | C | T |
| 2931 | 260869 | — | T |
| 2932 | 261010 | C | T |
| 2933 | 261037 | T | G |
| 2934 | 261079 | T | A |
| 2935 | 261133 | G | A |
| 2936 | 261141 | G | T |
| 2937 | 261171 | G | A |
| 2938 | 261254 | A | C |
| 2939 | 261456 | A | T |
| 2940 | 261483 | C | A |
| 2941 | 261570 | G | A |
| 2942 | 261639 | C | T |
| 2943 | 261967 | C | T |
| 2944 | 262135 | — | TT |
| 2945 | 262163 | T | C |
| 2946 | 262267 | T | C |
| 2947 | 262294 | G | A |
| 2948 | 262491 | T | C |
| 2949 | 262497 | C | T |
| 2950 | 262532 | T | G |
| 2951 | 262552 | C | T |
| 2952 | 262605 | T | A |
| 2953 | 262805 | C | T |
| 2954 | 262835 | A | C |
| 2955 | 262853 | — | GT |
| 2956 | 262976 | C | T |
| 2957 | 263015 | G | A |
| 2958 | 263021 | T | C |
| 2959 | 263084 | G | C |
| 2960 | 263140 | C | G |
| 2961 | 263150 | C | T |
| 2962 | 263212 | C | T |
| 2963 | 263296 | T | A |
| 2964 | 263341 | T | C |
| 2965 | 263343 | T | C |
| 2966 | 263387 | C | T |
| 2967 | 263514 | T | C |
| 2968 | 263552 | T | C |
| 2969 | 263686 | T | C |
| 2970 | 263821 | T | C |
| 2971 | 263847 | A | G |
| 2972 | 263930 | T | A |
| 2973 | 264117 | A | C |
| 2974 | 264172 | C | T |
| 2975 | 264316 | T | A |
| 2976 | 264349 | A | G |
| 2977 | 264379 | A | C |
| 2978 | 264511 | C | T |
| 2979 | 264518 | G | C |
| 2980 | 264521 | A | G |
| 2981 | 264534 | A | G |
| 2982 | 264592 | A | C |
| 2983 | 264682 | C | A |
| 2984 | 264724 | — | ATAT |
| 2985 | 264926 | — | T |
| 2986 | 265054 | C | T |
| 2987 | 265231 | A | G |
| 2988 | 265362 | G | C |
| 2989 | 265484 | T | A |
| 2990 | 265798 | T | C |
| 2991 | 265800 | G | A |
| 2992 | 265945 | C | T |
| 2993 | 266008 | G | C |
| 2994 | 266010 | C | G |
| 2995 | 266013 | G | A |
| 2996 | 266076 | T | C |
| 2997 | 266088 | A | T |
| 2998 | 266144 | G | A |
| 2999 | 266171 | G | A |
| 3000 | 266253 | — | TC |
| 3001 | 266333 | C | G |
| 3002 | 266421 | T | C |
| 3003 | 266436 | G | C |
| 3004 | 266477 | A | G |
| 3005 | 266503 | A | T |
| 3006 | 266523 | C | T |
| 3007 | 266570 | — | TGG |
| 3008 | 266596 | G | A |
| 3009 | 266613 | T | C |
| 3010 | 266628 | C | A |
| 3011 | 266639 | A | T |
| 3012 | 266662 | T | C |
| 3013 | 266702 | G | T |
| 3014 | 266707 | C | T |
| 3015 | 266733 | C | A |
| 3016 | 266804 | C | T |
| 3017 | 266849 | A | C |
| 3018 | 266880 | T | C |
| 3019 | 266943 | G | A |
| 3020 | 266999 | C | G |
| 3021 | 267030 | C | T |
| 3022 | 267090 | A | T |
| 3023 | 267113 | T | C |
| 3024 | 267163 | G | A |
| 3025 | 267196 | T | A |
| 3026 | 267213 | C | T |
| 3027 | 267226 | G | C |
| 3028 | 267257 | T | G |
| 3029 | 267341 | A | C |
| 3030 | 267349 | A | T |
| 3031 | 267355 | G | A |
| 3032 | 267359 | A | T |
| 3033 | 267375 | C | G |
| 3034 | 267443 | A | C |
| 3035 | 267491 | — | G |
| 3036 | 267503 | G | T |
| 3037 | 267544 | A | G |
| 3038 | 267629 | T | G |
| 3039 | 267654 | G | A |
| 3040 | 267674 | A | G |
| 3041 | 267711 | A | T |
| 3042 | 267712 | A | G |
| 3043 | 267717 | C | T |
| 3044 | 267725 | C | T |
| 3045 | 267766 | T | G |
| 3046 | 267776 | A | G |
| 3047 | 267815 | T | C |
| 3048 | 267822 | C | T |
| 3049 | 267863 | C | T |
| 3050 | 267869 | A | G |
| 3051 | 267896 | C | T |
| 3052 | 267976 | C | A |
| 3053 | 267981 | A | G |
| 3054 | 268004 | C | G |
| 3055 | 268107 | T | C |
| 3056 | 268115 | A | T |
| 3057 | 268117 | G | A |
| 3058 | 268195 | T | G |
| 3059 | 268235 | A | G |
| 3060 | 268255 | T | A |
| 3061 | 268266 | A | G |
| 3062 | 268308 | T | C |
| 3063 | 268337 | C | A |
| 3064 | 268369 | A | T |
| 3065 | 268370 | T | A |
| 3066 | 268421 | G | T |
| 3067 | 268436 | T | A |
| 3068 | 268480 | T | A |
| 3069 | 268494 | G | T |
| 3070 | 268519 | C | T |
| 3071 | 268520 | G | T |
| 3072 | 268658 | T | C |
| 3073 | 268893 | T | C |
| 3074 | 268979 | T | A |
| 3075 | 269014 | C | T |
| 3076 | 269044 | G | C |
| 3077 | 269140 | C | A |
| 3078 | 269153 | C | G |
| 3079 | 269627 | C | A |
| 3080 | 269662 | C | T |
| 3081 | 269665 | A | G |
| 3082 | 269680 | T | A |
| 3083 | 269784 | T | G |
| 3084 | 269968 | G | T |
| 3085 | 270089 | A | T |
| 3086 | 270132 | T | C |
| 3087 | 270182 | T | A |
| 3088 | 270235 | C | T |
| 3089 | 270288 | G | T |
| 3090 | 270362 | G | A |
| 3091 | 270418 | C | A |
| 3092 | 270441 | C | T |
| 3093 | 270690 | T | C |
| 3094 | 270700 | A | G |
| 3095 | 270744 | G | A |
| 3096 | 270864 | T | G |
| 3097 | 270872 | A | G |
| 3098 | 270885 | A | C |
| 3099 | 270971 | C | T |
| 3100 | 271092 | G | C |
| 3101 | 271301 | — | A |
| 3102 | 271387 | T | A |
| 3103 | 271399 | C | T |
| 3104 | 271617 | C | T |
| 3105 | 271632 | A | T |
| 3106 | 271645 | A | T |
| 3107 | 271648 | T | C |
| 3108 | 271652 | A | T |
| 3109 | 271710 | A | T |
| 3110 | 271827 | G | T |
| 3111 | 272005 | A | G |
| 3112 | 272080 | A | G |
| 3113 | 272131 | C | G |
| 3114 | 272176 | T | A |
| 3115 | 272226 | G | A |
| 3116 | 272576 | A | C |
| 3117 | 272597 | G | A |
| 3118 | 272711 | A | C |
| 3119 | 272721 | T | G |
| 3120 | 272724 | G | T |
| 3121 | 272725 | G | T |
| 3122 | 272736 | C | A |
| 3123 | 272754 | T | C |
| 3124 | 272800 | T | A |
| 3125 | 272809 | T | C |
| 3126 | 272818 | G | A |
| 3127 | 272954 | A | T |
| 3128 | 272988 | A | G |
| 3129 | 272989 | T | C |
| 3130 | 273100 | T | C |
| 3131 | 273135 | T | C |
| 3132 | 273175 | G | A |
| 3133 | 273264 | T | A |
| 3134 | 273283 | C | A |
| 3135 | 273328 | T | G |
| 3136 | 273350 | T | A |
| 3137 | 273402 | A | C |
| 3138 | 273432 | T | C |
| 3139 | 273434 | A | G |
| 3140 | 273480 | G | C |
| 3141 | 273514 | T | C |
| 3142 | 273539 | C | A |
| 3143 | 273559 | T | C |
| 3144 | 273680 | T | C |
| 3145 | 273806 | C | T |
| 3146 | 273939 | G | A |
| 3147 | 274035 | G | A |
| 3148 | 274096 | A | T |
| 3149 | 274117 | T | A |
| 3150 | 274196 | T | A |
| 3151 | 274234 | A | C |
| 3152 | 274249 | C | T |
| 3153 | 274303 | A | C |
| 3154 | 274322 | G | T |
| 3155 | 274352 | C | T |
| 3156 | 274363 | T | C |
| 3157 | 274387 | T | C |
| 3158 | 274395 | T | A |
| 3159 | 274508 | C | G |
| 3160 | 274570 | G | A |
| 3161 | 274594 | T | C |
| 3162 | 274728 | G | A |
| 3163 | 274836 | T | G |
| 3164 | 274911 | C | G |
| 3165 | 274926 | G | A |
| 3166 | 274928 | C | T |
| 3167 | 275023 | G | T |
| 3168 | 275066 | T | G |
| 3169 | 275071 | — | T |
| 3170 | 275108 | G | T |
| 3171 | 275264 | T | C |
| 3172 | 275273 | C | T |
| 3173 | 275284 | C | A |
| 3174 | 275305 | T | G |
| 3175 | 275378 | A | G |
| 3176 | 275381 | G | A |
| 3177 | 275438 | C | A |
| 3178 | 275448 | T | G |
| 3179 | 275867 | T | A |
| 3180 | 275890 | T | C |
| 3181 | 275922 | T | A |
| 3182 | 275923 | G | T |
| 3183 | 275939 | C | A |
| 3184 | 275966 | T | C |
| 3185 | 276037 | T | C |
| 3186 | 276146 | C | A |
| 3187 | 276149 | T | G |
| 3188 | 276164 | T | C |
| 3189 | 276178 | A | C |
| 3190 | 276229 | C | T |
| 3191 | 276300 | G | A |
| 3192 | 276303 | A | G |
| 3193 | 276532 | T | A |
| 3194 | 276542 | G | A |
| 3195 | 276560 | T | C |
| 3196 | 276583 | A | T |
| 3197 | 276640 | C | G |
| 3198 | 276643 | C | T |
| 3199 | 276649 | C | G |
| 3200 | 276726 | C | T |
| 3201 | 276794 | T | C |
| 3202 | 276932 | C | T |
| 3203 | 276956 | A | C |
| 3204 | 277008 | A | T |
| 3205 | 277021 | A | T |
| 3206 | 277053 | C | T |
| 3207 | 277161 | T | A |
| 3208 | 277184 | C | T |
| 3209 | 277186 | A | G |
| 3210 | 278578 | G | A |
| 3211 | 278745 | C | T |
| 3212 | 278746 | T | A |
| 3213 | 278753 | A | T |
| 3214 | 279105 | C | T |
| 3215 | 279112 | T | C |
| 3216 | 279130 | A | G |
| 3217 | 279165 | C | A |
| 3218 | 279233 | T | A |
| 3219 | 279487 | T | C |
| 3220 | 279495 | C | T |
| 3221 | 279704 | T | A |
| 3222 | 279718 | A | C |
| 3223 | 279773 | T | A |
| 3224 | 279799 | A | G |
| 3225 | 279840 | G | A |
| 3226 | 279914 | T | C |
| 3227 | 279922 | T | G |
| 3228 | 279935 | C | A |
| 3229 | 280137 | G | A |
| 3230 | 280179 | C | T |
| 3231 | 280240 | A | G |
| 3232 | 280254 | G | A |
| 3233 | 280278 | C | G |
| 3234 | 280300 | C | A |
| 3235 | 280306 | G | T |
| 3236 | 280317 | C | G |
| 3237 | 280330 | G | A |
| 3238 | 280336 | G | T |
| 3239 | 280337 | G | C |
| 3240 | 280371 | G | A |
| 3241 | 280378 | C | T |
| 3242 | 280425 | G | A |
| 3243 | 280543 | G | A |
| 3244 | 280547 | T | C |
| 3245 | 280557 | T | G |
| 3246 | 280575 | C | G |
| 3247 | 280614 | C | A |
| 3248 | 280617 | G | T |
| 3249 | 280651 | T | A |
| 3250 | 280652 | T | C |
| 3251 | 280658 | G | A |
| 3252 | 280662 | T | A |
| 3253 | 280732 | T | A |
| 3254 | 280733 | T | C |
| 3255 | 280748 | C | A |
| 3256 | 280862 | C | G |
| 3257 | 281479 | C | G |
| 3258 | 281481 | A | C |
| 3259 | 281493 | G | C |
| 3260 | 281639 | C | G |
| 3261 | 281640 | A | G |
| 3262 | 282948 | T | C |
| 3263 | 287002 | A | T |
| 3264 | 287003 | T | A |
| 3265 | 287101 | T | C |
| 3266 | 287108 | G | A |
| 3267 | 287141 | C | G |
| 3268 | 287144 | G | A |
| 3269 | 287149 | C | T |
| 3270 | 287275 | G | A |
| 3271 | 287344 | A | T |
| 3272 | 287381 | T | A |
| 3273 | 287393 | T | C |
| 3274 | 287397 | T | C |
| 3275 | 287529 | T | C |
| 3276 | 287545 | A | T |
| 3277 | 287557 | A | C |
| 3278 | 287607 | T | C |
| 3279 | 287641 | T | G |
| 3280 | 287945 | G | C |
| 3281 | 287969 | A | G |
| 3282 | 287985 | A | G |
| 3283 | 288018 | G | C |
| 3284 | 288092 | C | A |
| 3285 | 288095 | G | A |
| 3286 | 288102 | T | C |
| 3287 | 288643 | G | T |
| 3288 | 288646 | C | G |
| 3289 | 288654 | G | A |
| 3290 | 288655 | A | G |
| 3291 | 288660 | G | A |
| 3292 | 288671 | T | C |
| 3293 | 288672 | C | A |
| 3294 | 288830 | G | A |
| 3295 | 288836 | G | A |
| 3296 | 288870 | G | C |
| 3297 | 288871 | C | A |
| 3298 | 288886 | A | C |
| 3299 | 288943 | C | T |
| 3300 | 288962 | G | A |
| 3301 | 288970 | G | A |
| 3302 | 289009 | C | T |
| 3303 | 289037 | T | G |
| 3304 | 289038 | C | G |
| 3305 | 289156 | G | C |
| 3306 | 289162 | C | G |
| 3307 | 289264 | A | T |
| 3308 | 289281 | G | C |
| 3309 | 289284 | A | G |
| 3310 | 289526 | G | A |
| 3311 | 289567 | G | T |
| 3312 | 289575 | A | G |
| 3313 | 289598 | C | A |
| 3314 | 289630 | A | T |
| 3315 | 289703 | G | A |
| 3316 | 289745 | A | T |
| 3317 | 289764 | T | A |
| 3318 | 289792 | G | T |
| 3319 | 289862 | G | T |
| 3320 | 289877 | A | C |
| 3321 | 289955 | T | G |
| 3322 | 289977 | T | G |
| 3323 | 290045 | A | T |
| 3324 | 290046 | T | A |
| 3325 | 290054 | C | T |
| 3326 | 290098 | A | T |
| 3327 | 290292 | A | G |
| 3328 | 291161 | T | C |
| 3329 | 291386 | T | A |
| 3330 | 292155 | G | A |
| 3331 | 292466 | G | A |
| 3332 | 292971 | A | C |
| 3333 | 293726 | T | C |
| 3334 | 293919 | A | T |
| 3335 | 294541 | A | C |
| 3336 | 294598 | G | A |
| 3337 | 294649 | T | C |
| 3338 | 294697 | A | G |
| 3339 | 295680 | C | A |
| 3340 | 296385 | C | A |
| 3341 | 296649 | A | G |
| 3342 | 296656 | G | T |
| 3343 | 296812 | C | T |
| 3344 | 296832 | C | A |
| 3345 | 296869 | C | A |
| 3346 | 297026 | G | A |
| 3347 | 297042 | T | C |
| 3348 | 297605 | T | C |
| 3349 | 297697 | T | C |
| 3350 | 298499 | T | A |
| 3351 | 299112 | A | C |
| 3352 | 301248 | T | C |
| 3353 | 301502 | T | G |
| 3354 | 302448 | A | G |
| 3355 | 302653 | A | G |
| 3356 | 302711 | G | T |
| 3357 | 302917 | C | A |
| 3358 | 303370 | T | C |
| 3359 | 303828 | G | A |
| 3360 | 304193 | A | C |
| 3361 | 304638 | A | G |
| 3362 | 305141 | T | C |
| 3363 | 305373 | T | C |
| 3364 | 305441 | A | G |
| 3365 | 305529 | T | G |
| 3366 | 305554 | T | C |
| 3367 | 306016 | G | C |
| 3368 | 306697 | A | G |
| 3369 | 307059 | G | T |
| 3370 | 307091 | T | C |
| 3371 | 307111 | A | T |
| 3372 | 307161 | A | T |
| 3373 | 307188 | A | G |
| 3374 | 307384 | T | A |
| 3375 | 307547 | A | T |
| 3376 | 307672 | T | G |
| 3377 | 307698 | C | T |
| 3378 | 307741 | A | C |
| 3379 | 307744 | A | G |
| 3380 | 307809 | A | G |
| 3381 | 307905 | C | A |
| 3382 | 308028 | G | T |
| 3383 | 308164 | C | G |
| 3384 | 308185 | A | G |
| 3385 | 308535 | A | G |
| 3386 | 308574 | G | T |
| 3387 | 308583 | G | A |
| 3388 | 308657 | C | G |
| 3389 | 308722 | T | C |
| 3390 | 308728 | T | A |
| 3391 | 308953 | T | C |
| 3392 | 309025 | G | A |
| 3393 | 309116 | A | G |
| 3394 | 309369 | A | G |
| 3395 | 309501 | G | A |
| 3396 | 309577 | C | T |
| 3397 | 309626 | A | T |
| 3398 | 309637 | A | G |
| 3399 | 309768 | T | A |
| 3400 | 309800 | C | T |
| 3401 | 309877 | A | G |
| 3402 | 310022 | G | A |
| 3403 | 310025 | G | A |
| 3404 | 310039 | T | C |
| 3405 | 310084 | T | C |
| 3406 | 310119 | T | C |
| 3407 | 310123 | C | T |
| 3408 | 310151 | C | A |
| 3409 | 310185 | T | C |
| 3410 | 310197 | C | A |
| 3411 | 310464 | T | C |
| 3412 | 310703 | G | A |
| 3413 | 310922 | C | A |
| 3414 | 310933 | C | T |
| 3415 | 310946 | T | C |
| 3416 | 310970 | T | A |
| 3417 | 311010 | T | C |
| 3418 | 311137 | T | G |
| 3419 | 311183 | A | G |
| 3420 | 311184 | A | G |
| 3421 | 311473 | A | G |
| 3422 | 311857 | G | C |
| 3423 | 311937 | A | C |
| 3424 | 312580 | A | C |
| 3425 | 312684 | A | T |
| 3426 | 312749 | T | A |
| 3427 | 313009 | G | A |
| 3428 | 313307 | G | A |
| 3429 | 313340 | — | G |
| 3430 | 313491 | A | C |
| 3431 | 313550 | C | A |
| 3432 | 313648 | G | A |
| 3433 | 314143 | C | T |
| 3434 | 314305 | G | C |
| 3435 | 314384 | G | A |
| 3436 | 314504 | T | C |
| 3437 | 314703 | C | A |
| 3438 | 314810 | T | A |
| 3439 | 314821 | A | T |
| 3440 | 315163 | G | T |
| 3441 | 315443 | G | A |
| 3442 | 315514 | C | A |
| 3443 | 315611 | T | G |
| 3444 | 315634 | T | C |
| 3445 | 315684 | A | G |
| 3446 | 315694 | T | C |
| 3447 | 315855 | C | T |
| 3448 | 316534 | G | A |
| 3449 | 316594 | G | A |
| 3450 | 316843 | — | ACAGCAAT |
| 3451 | 317340 | T | C |
| 3452 | 317641 | C | T |
| 3453 | 317993 | A | G |
| 3454 | 318084 | A | G |
| 3455 | 318212 | T | C |
| 3456 | 318451 | T | C |
| 3457 | 318465 | T | A |
| 3458 | 318638 | C | G |
| 3459 | 318893 | T | A |
| 3460 | 319458 | T | A |
| 3461 | 319546 | G | A |
| 3462 | 319620 | A | G |
| 3463 | 319626 | T | G |
| 3464 | 319790 | A | T |
| 3465 | 319994 | A | C |
| 3466 | 320393 | A | G |
| 3467 | 321238 | T | C |
| 3468 | 321457 | T | A |
| 3469 | 321495 | A | C |
| 3470 | 321558 | A | T |
| 3471 | 321559 | A | T |
| 3472 | 321560 | A | T |
| 3473 | 321753 | A | C |
| 3474 | 321873 | T | C |
| 3475 | 322481 | G | A |
| 3476 | 322998 | T | G |
| 3477 | 323155 | T | A |
| 3478 | 323358 | T | C |
| 3479 | 323461 | A | G |
| 3480 | 323489 | A | C |
| 3481 | 323825 | G | A |
| 3482 | 323865 | A | C |
| 3483 | 323923 | A | G |
| 3484 | 324280 | G | A |
| 3485 | 324443 | — | A |
| 3486 | 325103 | G | C |
| 3487 | 325282 | T | C |
| 3488 | 325284 | C | T |
| 3489 | 325307 | G | A |
| 3490 | 325683 | T | A |
| 3491 | 326047 | G | A |
| 3492 | 326396 | G | T |
| 3493 | 326443 | C | T |
| 3494 | 326530 | A | G |
| 3495 | 326625 | G | A |
| 3496 | 326701 | A | G |
| 3497 | 326996 | T | C |
| 3498 | 327039 | G | A |
| 3499 | 327161 | C | T |
| 3500 | 327259 | A | G |
| 3501 | 327315 | C | T |
| 3502 | 327357 | C | T |
| 3503 | 327399 | A | G |
| 3504 | 327431 | G | A |
| 3505 | 327444 | G | A |
| 3506 | 327488 | T | C |
| 3507 | 327490 | G | T |
| 3508 | 327598 | C | T |
| 3509 | 327625 | A | G |
| 3510 | 327665 | T | C |
| 3511 | 327736 | A | G |
| 3512 | 327877 | C | T |
| 3513 | 327879 | T | C |
| 3514 | 327911 | G | A |
| 3515 | 327962 | G | T |
| 3516 | 328023 | T | G |
| 3517 | 328043 | G | C |
| 3518 | 328067 | G | A |
| 3519 | 328089 | G | A |
| 3520 | 328125 | G | A |
| 3521 | 328198 | A | G |
| 3522 | 328404 | C | T |
| 3523 | 328414 | C | G |
| 3524 | 328598 | G | A |
| 3525 | 328672 | C | T |
| 3526 | 328711 | A | G |
| 3527 | 328892 | A | C |
| 3528 | 328913 | A | G |
| 3529 | 329013 | C | T |
| 3530 | 329042 | C | T |
| 3531 | 329171 | G | A |
| 3532 | 329183 | T | C |
| 3533 | 329210 | T | C |
| 3534 | 329216 | T | C |
| 3535 | 329261 | A | T |
| 3536 | 329396 | A | G |
| 3537 | 329426 | G | C |
| 3538 | 329526 | T | C |
| 3539 | 329534 | T | C |
| 3540 | 329678 | G | A |
| 3541 | 329725 | C | T |
| 3542 | 329795 | G | A |
| 3543 | 329820 | G | T |
| 3544 | 329846 | C | T |
| 3545 | 329854 | A | T |
| 3546 | 329859 | A | G |
| 3547 | 330048 | T | C |
| 3548 | 330075 | A | G |
| 3549 | 330084 | T | A |
| 3550 | 330172 | T | C |
| 3551 | 330339 | A | G |
| 3552 | 330386 | C | T |
| 3553 | 330414 | C | G |
| 3554 | 333419 | G | T |
| 3555 | 333520 | G | A |
| 3556 | 333544 | A | G |
| 3557 | 333594 | C | T |
| 3558 | 333616 | C | T |
| 3559 | 333666 | A | G |
| 3560 | 334066 | C | T |
| 3561 | 334138 | C | T |
| 3562 | 334210 | G | A |
| 3563 | 334247 | C | T |
| 3564 | 334315 | T | C |
| 3565 | 334333 | A | G |
| 3566 | 334364 | A | G |
| 3567 | 334434 | T | G |
| 3568 | 334521 | C | T |
| 3569 | 334655 | A | G |
| 3570 | 334680 | C | A |
| 3571 | 334798 | T | C |
| 3572 | 334829 | C | A |
| 3573 | 334873 | A | G |
| 3574 | 334938 | G | A |
| 3575 | 334972 | T | C |
| 3576 | 335053 | T | A |
| 3577 | 335085 | G | A |
| 3578 | 335202 | G | A |
| 3579 | 335367 | A | C |
| 3580 | 335426 | T | A |
| 3581 | 335435 | A | T |
| 3582 | 335460 | A | G |
| 3583 | 335471 | C | T |
| 3584 | 335499 | T | C |
| 3585 | 335514 | A | G |
| 3586 | 335572 | G | A |
| 3587 | 335642 | C | T |
| 3588 | 335690 | G | A |
| 3589 | 335720 | A | C |
| 3590 | 335765 | T | C |
| 3591 | 335895 | G | T |
| 3592 | 335900 | A | G |
| 3593 | 336015 | T | C |
| 3594 | 336122 | C | T |
| 3595 | 336132 | C | T |
| 3596 | 336454 | A | G |
| 3597 | 336468 | G | A |
| 3598 | 336487 | G | A |
| 3599 | 336596 | G | A |
| 3600 | 336608 | T | C |
| 3601 | 336686 | C | T |
| 3602 | 336694 | T | G |
| 3603 | 336725 | A | G |
| 3604 | 336733 | G | A |
| 3605 | 336865 | C | T |
| 3606 | 336917 | — | G |
| 3607 | 336946 | C | T |
| 3608 | 337076 | C | T |
| 3609 | 337113 | A | T |
| 3610 | 337215 | — | C |
| 3611 | 337295 | G | A |
| 3612 | 337320 | A | C |
| 3613 | 337539 | C | A |
| 3614 | 337635 | A | C |
| 3615 | 337929 | T | C |
| 3616 | 337949 | A | G |
| 3617 | 338017 | G | A |
| 3618 | 338085 | G | A |
| 3619 | 338111 | G | A |
| 3620 | 338132 | A | G |
| 3621 | 338151 | C | T |
| 3622 | 338234 | A | G |
| 3623 | 338256 | A | C |
| 3624 | 338340 | A | G |
| 3625 | 338360 | T | G |
| 3626 | 338400 | G | A |
| 3627 | 338552 | A | G |
| 3628 | 338918 | G | A |
| 3629 | 339098 | — | C |
| 3630 | 339192 | A | G |
| 3631 | 339214 | G | A |
| 3632 | 339227 | G | A |
| 3633 | 339309 | A | G |
| 3634 | 339391 | C | G |
| 3635 | 339532 | T | C |
| 3636 | 339714 | A | G |
| 3637 | 339985 | — | AAT |
| 3638 | 340186 | T | C |
| 3639 | 340731 | G | A |
| 3640 | 341366 | T | C |
| 3641 | 341953 | T | A |
| 3642 | 342028 | G | A |
| 3643 | 342087 | A | T |
| 3644 | 342861 | T | C |
| 3645 | 343102 | G | T |
| 3646 | 343425 | G | A |
| 3647 | 343468 | T | A |
| 3648 | 343475 | G | A |
| 3649 | 343484 | T | A |
| 3650 | 343548 | T | C |
| 3651 | 343866 | A | T |
| 3652 | 344261 | C | T |
| 3653 | 344318 | C | G |
| 3654 | 344545 | A | C |
| 3655 | 344884 | C | T |
| 3656 | 345059 | A | C |

It is well known in the art that given the sequence and the SNP allele associated with a given trait (e.g. ASR resistance), one having ordinary skill in the art could develop oligonucleotide primers and use said primers to identify plants carrying the chromosomal interval of SEQ ID NO: 1, or a portion thereof. In example embodiments, a TAQMAN® assay (e.g. generally a two-step allelic discrimination assay or similar), a KASP™ assay (generally a one-step allelic discrimination assay defined below or similar), or both can be employed to assay one or more of the SNPs disclosed in Table 1. In an exemplary two-step assay, a forward primer, a reverse primer, and two assay probes (or hybridization oligos) are employed. The forward and reverse primers are employed to amplify genetic loci that comprise SNPs that are associated with ASR resistance loci. The particular nucleotides that are present at the SNP positions are then assayed using the assay primers (which in some embodiments are differentially labeled with, for example, fluorophores to permit distinguishing between the two assay probes in a single reaction), which in each pair differ from each other with respect to the nucleotides that are present at the SNP position (although it is noted that in any given pair, the probes can differ in their 5′ or 3′ ends without impacting their abilities to differentiate between nucleotides present at the corresponding SNP positions). In some embodiments, the assay primers and the reaction conditions are designed such that an assay primer will only hybridize to the reverse complement of a 100% perfectly matched sequence, thereby permitting identification of which allele(s) is/are present based upon detection of hybridizations. Example primers and probes are provided herein with reference to Example 3 and Table 3.

### Genetic Mapping

Genetic loci correlating with particular phenotypes, such as disease resistance, can be mapped in an organism's genome. By identifying a marker or cluster of markers that co-segregate with a trait of interest, the breeder is able to rapidly select a desired phenotype by selecting for the proper marker (a process called marker-assisted selection, or MAS). Such markers may also be used by breeders to design genotypes in silico and to practice whole genome selection.

The present invention provides markers associated with enhanced disease resistance. Detection of these markers and/or other linked markers can be used to identify, select and/or produce Disease resistant plants and/or to eliminate plants that are not Disease resistant from breeding programs or planting.

Glycine canescens Genetic Loci Associated with Enhanced Disease Resistance

Chromosome intervals are provided herein that are associated with enhanced disease resistance. When introgressed into the genome of a plant, the introgressed chromosomal interval confers the plant with enhanced disease resistance. In particular embodiments, the chromosomal interval comprises SEQ ID NO: 1, or a portion thereof. In particular embodiments, the chromosomal interval comprising SEQ ID NO: 1, or a portion thereof, confers enhanced resistance to Asian Soy Rust (ASR). In particular embodiments, the chromosomal interval comprising SEQ ID NO: 1, or a portion thereof, that confers enhanced resistance to Asian Soy Rust (ASR), is derived from Glycine canescens, such as from Glycine canescens Accession line PI446934, or a progeny thereof.

As used herein, “chromosome interval” designates a contiguous linear span of genomic DNA that resides in planta on a single chromosome. The term also designates any and all genomic intervals defined by any of the markers set forth in this invention. The genetic elements located on a single chromosome interval are physically linked and the size of a chromosome interval is not particularly limited. In some aspects, the genetic elements located within a single chromosome interval are genetically linked, typically with a genetic recombination distance of, for example, less than or equal to 20 cM, less than or equal to 10 cM, or less than or equal to 5 cM. That is, two genetic elements within a single chromosome interval undergo meiotic recombination at a frequency of less than or equal to 20% or 10% or 5%, respectively.

As used herein, the portion of the chromosomal interval of SEQ ID NO: 1 comprises at least 10%; at least 11%; at least 12%; at least 13%; at least 14%; at least 15%; at least 16%%; at least 17%; at least 18%; at least 19%; at least 20%; at least 21%; at least 22%; at least 23%; at least 24%; at least 25%; at least 26%; at least 27%; at least 28%; at least 29%; at least 30%; at least 31%; at least 32%; at least 33%; at least 34%; at least 35%; at least 36%; at least 37%; at least 38%; a t least 39%; at least 40%; at least 41%; at least 42%; at least 43%; at least 44%; at least 45%; at least 46%; at least %; at least 48%; at least 49%; or at least 50%; at least 51%; at least 52%; at least 53%; at least 54%; at least 55%; at least 56%; at least 57%; at least 58%; at least 59%; or at least 60%; at least 61%; at least 62%; at least 63%; at least 64%; at least 65%; at least 66%; at least 67%; at least 68%; at least 69%; at least 70%; at least 71%; at least 72%; at least 73%; at least 74%; at least 75%; at least 76%; at least 77%; at least 78%; at least 79%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; or at least 99% of SEQ ID NO: 1.

The boundaries of a chromosome interval can be defined by genetic recombination distance or by markers. In one embodiment, the boundaries of a chromosome interval comprise markers. In another embodiment, the boundaries of a chromosome interval comprise markers that are linked to a gene controlling the trait of interest, i.e., any marker that lies within a given interval, including the terminal markers that define the boundaries of the interval, and that can be used as a marker for the presence or absence of disease resistance.

In particular embodiments, the chromosomal interval that confers enhanced disease resistance (e.g., enhanced ASR resistance) comprises SEQ ID NO: 1, or a portion thereof that is flanked by SNP Marker No. 1850 and SNP Marker No. 3656 of Table 1. In other particular embodiments, the chromosomal interval that confers enhanced ASR resistance comprises SEQ ID NO: 1, or a portion thereof that is flanked by SNP Marker No. 3115 and SNP Marker No. 3347 of Table 1. In still other embodiments, the chromosomal interval comprises SEQ ID NO: 1 or a portion thereof one or more of the favorable markers of Table 1.

An interval, or portion thereof, described by the markers that flank the interval include the indicated flanking markers and any marker localizing within that interval, whether those markers are currently known or unknown. Although it is anticipated that one skilled in the art may describe additional polymorphic sites at marker loci in and around the markers identified herein, any marker within the chromosome intervals described herein that are associated with disease resistance fall within the scope of this claimed invention. In example embodiments, the chromosomal interval that confers enhanced disease resistance (e.g., enhanced ASR resistance) comprises SEQ ID NO: 1, or a portion of SEQ ID NO: 1 that is flanked by SNP Marker No. 1850 and SNP Marker No. 3656 of Table 1, or a portion of SEQ ID NO: 1 comprising SNP Marker No. 1850 and/or SNP Marker No. 3656 of Table 1, and/or any of the markers located between SNP Marker No. 1850 and SNP Marker No. 3656 of Table 1. In other example embodiments, the chromosomal interval that confers enhanced disease resistance (e.g., enhanced ASR resistance) comprises SEQ ID NO: 1, or a portion of SEQ ID NO: 1 that is flanked by SNP Marker No. 3115 and SNP Marker No. 3347 of Table 1, or a portion of SEQ ID NO: 1 comprising SNP Marker No. 3115 and/or SNP Marker No. 3347 of Table 1, and/or any of the markers located between SNP Marker No. 3115 and SNP Marker No. 3347 of Table 1.

“Quantitative trait loci” or a “quantitative trait locus” (QTL), as used herein, is a genetic interval that effects a phenotype that can be described in quantitative terms and can be assigned a “phenotypic value” which corresponds to a quantitative value for the phenotypic trait. A QTL can act through a single gene mechanism or by a polygenic mechanism. In some aspects, the invention provides QTL chromosome intervals, where a QTL (or multiple QTLs) that segregates with disease resistance is contained in those intervals. In one embodiment of this invention, the boundaries of chromosome intervals are drawn to encompass markers that will be linked to one or more QTL. In other words, the chromosome interval is drawn such that any marker that lies within that interval (including the terminal markers that define the boundaries of the interval) is genetically linked to the QTL. Each chromosomal interval comprises at least one QTL.

Markers associated with enhanced disease resistance are identified herein at Table 1. A marker of the present invention may comprise a single allele or a combination of alleles at one or more genetic loci (for example, any combination of markers from Table 1). In particular embodiments, the marker may comprise one or more marker alleles located within a chromosomal interval comprising SEQ ID NO: 1, or a portion thereof, that are associated with ASR resistance, particularly any of the favorable markers of Table 1. In other particular embodiments, the marker may comprise any marker located between position 1 and position 527424 of SEQ ID NO: 1.

In one example embodiment, the marker of the present invention is SNP marker 1850 of Table 1, or any marker associated with ASR located within 20 cM, 10 cM, 5 cM, 4 cM, 3 cM, 2 cM, 1 cM, 0.5 cM or 0.1 cM of SNP marker 1850 of Table 1. In another example embodiment, the marker of the present invention is SNP marker 3656 of Table 1, or any marker associated with ASR located within 20 cM, 10 cM, 5 cM, 4 cM, 3 cM, 2 cM, 1 cM, 0.5 cM or 0.1 cM of SNP marker 3656 of Table 1. In still another embodiment, the marker of the present invention is any marker associated with the chromosomal interval of SEQ ID NO: 1 that is located between SNP marker 1850 and SNP marker 3656 of Table 1, or any marker located within 20 cM, 10 cM, 5 cM, 4 cM, 3 cM, 2 cM, 1 cM, 0.5 cM or 0.1 cM thereof.

In other particular embodiments, the marker of the present invention is SNP marker 3115 of Table 1, or any marker associated with ASR located within 20 cM, 10 cM, 5 cM, 4 cM, 3 cM, 2 cM, 1 cM, 0.5 cM or 0.1 cM of SNP marker 3115 of Table 1. In another example embodiment, the marker of the present invention is SNP marker 3347 of Table 1, or any marker associated with ASR located within 20 cM, 10 cM, 5 cM, 4 cM, 3 cM, 2 cM, 1 cM, 0.5 cM or 0.1 cM of SNP marker 3347 of Table 1. In still another embodiment, the marker of the present invention is any marker associated with the chromosomal interval of SEQ ID NO: 1 that is located between SNP marker 3115 and SNP marker 3347 of Table 1, or any marker located within 20 cM, 10 cM, 5 cM, 4 cM, 3 cM, 2 cM, 1 cM, 0.5 cM or 0.1 cM thereof.

In still further embodiments, the marker of the present invention is a SNP marker located at or between positions 152,261 and 345,059 of SEQ ID NO: 1, such as at or between positions 272,226 and 297,561 of SEQ ID NO: 1. In still other embodiments, the marker of the present invention is any marker associated ASR resistance that is within 20 cM, 10 cM, 5 cM, 4 cM, 3 cM, 2 cM, 1 cM, 0.5 cM or 0.1 cM of a SNP marker located at or between positions 152,261 and 345,059 of SEQ ID NO: 1, such as a SNP marker within 20 cM, 10 cM, 5 cM, 4 cM, 3 cM, 2 cM, 1 cM, 0.5 cM or 0.1 cM of a SNP marker located at or between positions 272,226 and 297,561 of SEQ ID NO: 1.

Markers of the present invention are described herein with respect to the positions of marker loci within the chromosomal interval comprising sequenced genomic DNA of PI446934, or a progeny thereof as depicted by SEQ ID NO: 1 and as represented in Table 1.

When referring to marker positions in cM, position zero corresponds to the first (most distal) marker known at the beginning of the chromosome on both Syngenta's internal consensus genetic map and the Williams 82 v2.0 reference genomic map, which is freely available to the public from the soybase(.)org website.

### Marker-Assisted Selection

Markers can be used in a variety of plant breeding applications. See, e.g., Staub et al., Hortscience 31: 729 (1996); Tanksley, Plant Molecular Biology Reporter 1: 3 (1983). One of the main areas of interest is to increase the efficiency of backcrossing and introgressing genes using marker-assisted selection (MAS). In general, MAS takes advantage of genetic markers that have been identified as having a significant likelihood of co-segregation with a desired trait. Such markers are presumed to be in/near the gene(s) that give rise to the desired phenotype, and their presence indicates that the plant will possess the desired trait. Plants which possess the marker are expected to transfer the desired phenotype to their progeny.

A marker that demonstrates linkage with a locus affecting a desired phenotypic trait provides a useful tool for the selection of the trait in a plant population. This is particularly true where the phenotype is hard to assay or occurs at a late stage in plant development. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing or imparting the trait. Having flanking markers decreases the chances that false positive selection will occur. The ideal situation is to have a marker within the causative gene itself, so that recombination cannot occur between the marker and the gene. Such a marker is called a “perfect marker.”

When a gene is introgressed by MAS, it is not only the gene that is introduced but also the flanking regions. Gepts, Crop Sci 42:1780 (2002). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite soybean line. This is also sometimes referred to as “yield drag.” The size of the flanking region can be decreased by additional backcrossing, although this is not always successful, as breeders do not have control over the size of the region or the recombination breakpoints. Young et al., Genetics 120:579 (1998). In classical breeding, it is usually only by chance that recombinations that contribute to a reduction in the size of the donor segment are selected. Tanksley et al., Biotechnology 7: 257 (1989). Even after 20 backcrosses, one might find a sizeable piece of the donor chromosome still linked to the gene being selected. With markers, however, it is possible to select those rare individuals that have experienced recombination near the gene of interest. In 150 backcross plants, there is a 95% chance that at least one plant will have experienced a crossover within 1 cM of the gene, based on a single meiosis map distance. Markers allow for unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers. See Tanksley et al., supra. When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of integrated linkage maps of the soybean genome containing increasing densities of public soybean markers has facilitated soybean genetic mapping and MAS.

Of all the molecular marker types, SNPs are the most abundant and have the potential to provide the highest genetic map resolution. Bhattramakki et al., Plant Molec. Biol. 48:539 (2002). SNPs can be assayed in a so-called “ultra-high-throughput” fashion because they do not require large amounts of nucleic acid and automation of the assay is straight-forward. SNPs also have the benefit of being relatively low-cost systems. These three factors together make SNPs highly attractive for use in MAS. Several methods are available for SNP genotyping, including but not limited to hybridization, primer extension, oligonucleotide ligation, nuclease cleavage, mini-sequencing and coded spheres. Such methods have been reviewed in various publications: Gut, Hum. Mutat. 17:475 (2001); Shi, Clin. Chem. 47:164 (2001); Kwok, Pharmacogenomics 1:95 (2000); Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode™ (Qiagen, Germantown, MD), Invader® (Hologic, Madison, WI), SnapShot® (Applied Biosystems, Foster City, CA), Tagman® (Applied Biosystems, Foster City, CA) and Beadarrays™ (Illumina, San Diego, CA).

A number of SNP alleles together within a sequence, or across linked sequences, can be used to describe a haplotype for any particular genotype. Ching et al., BMC Genet. 3:19 (2002); Gupta et al., (2001), Rafalski, Plant Sci. 162:329 (2002b). Haplotypes can be more informative than single SNPs and can be more descriptive of any particular genotype. For example, a single SNP may be allele “T” for a specific Disease resistant line or variety, but the allele “T” might also occur in the soybean breeding population being utilized for recurrent parents. In this case, a combination of alleles at linked SNPs may be more informative. Once a unique haplotype has been assigned to a donor chromosomal region, that haplotype can be used in that population or any subset thereof to determine whether an individual has a particular gene. The use of automated high throughput marker detection platforms known to those of ordinary skill in the art makes this process highly efficient and effective.

The markers of the present invention can be used in marker-assisted selection protocols to identify and/or select progeny with enhanced Asian soybean rust tolerance. Such methods can comprise, consist essentially of or consist of crossing a first soybean plant or germplasm with a second soybean plant or germplasm, wherein the first soybean plant or germplasm comprises, in its genome, a chromosomal interval conferring ASR resistance, the chromosomal interval comprising SEQ ID NO: 1, or a portion thereof, and selecting a progeny plant that possesses a marker associated with the chromosomal interval of SEQ ID NO: 1, or the portion thereof. In particular, the chromosome interval comprises at least one allele as depicted in Table 1 and presence of the chromosomal interval in the progeny plant is detected by detecting for the presence of a favorable allele of any of the markers of Table 1.

Either of the first and second soybean plants, or both, may be of a non-naturally occurring variety of soybean. In some embodiments, the second soybean plant or germplasm is of an elite variety of soybean. In some embodiments, the genome of the second soybean plant or germplasm is at least about 50%, 55%, 60%, 65%, 70%, 75%, 80%, 85%, 90%, 95%, 97%, 99% or 100% identical to that of an elite variety of soybean.

Methods for identifying and/or selecting a disease resistant soybean plant or germplasm may comprise, consist essentially of or consist of detecting the presence of a marker associated with enhanced ASR tolerance. The marker may be detected in any sample taken from the plant or germplasm, including, but not limited to, the whole plant or germplasm, a portion of said plant or germplasm (e.g., a seed chip, a leaf punch disk or a cell from said plant or germplasm) or a nucleotide sequence from said plant or germplasm. Such a sample may be taken from the plant or germplasm using any present or future method known in the art, including, but not limited to, automated methods of removing a portion of endosperm with a sharp blade, drilling a small hole in the seed and collecting the resultant powder, cutting the seed with a laser and punching a leaf disk. The soybean plant may be of a non-naturally occurring variety of soybean. In some embodiments, the genome of the soybean plant or germplasm is at least about 50%, 55%, 60%, 65%, 70%, 75%, 80%, 85%, 90%, 95%, 97%, 99% or 100% identical to that of an elite variety of soybean.

In some embodiments, the marker detected in the sample may comprise, consist essentially of or consist of one or more marker alleles located within the chromosomal interval selected from a chromosomal interval derived from PI446934 or a progeny thereof wherein said chromosomal interval comprises SEQ ID NO: 1; or a portion thereof.

In example embodiments, methods of identifying and/or selecting plants having enhanced ASR resistance comprise detecting in the genome of a plant, presence of (i) SNP marker 1850 of Table 1, and/or any marker associated with ASR located within 20 cM, 10 cM, 5 cM, 4 cM, 3 cM, 2 cM, 1 cM, 0.5 cM or 0.1 cM of SNP marker 1850 of Table 1; (ii) SNP marker 3656 of Table 1, and/or any marker associated with ASR located within 20 cM, 10 cM, 5 cM, 4 cM, 3 cM, 2 cM, 1 cM, 0.5 cM or 0.1 cM of SNP marker 3656 of Table 1; (iii) at least one SNP marker associated with the chromosomal interval of SEQ ID NO: 1 that is/are located between SNP marker 1850 and SNP marker 3656 of Table 1, and/or any marker located within 20 cM, 10 cM, 5 cM, 4 cM, 3 cM, 2 cM, 1 cM, 0.5 cM or 0.1 cM thereof; (iv) SNP marker 3115 of Table 1, and/or any marker associated with ASR located within 20 cM, 10 cM, 5 cM, 4 cM, 3 cM, 2 cM, 1 cM, 0.5 cM or 0.1 cM of SNP marker 3115 of Table 1; (v) SNP marker 3347 of Table 1, and/or any marker associated with ASR located within 20 cM, 10 cM, 5 cM, 4 cM, 3 cM, 2 cM, 1 cM, 0.5 cM or 0.1 cM of SNP marker 3347 of Table 1; and/or (vi) at least one marker associated with the chromosomal interval of SEQ ID NO: 1 that is located between SNP marker 3115 and SNP marker 3347 of Table 1, and/or any marker located within 20 cM, 10 cM, 5 cM, 4 cM, 3 cM, 2 cM, 1 cM, 0.5 cM or 0.1 cM thereof.

In still further embodiments, methods of identifying and/or selecting plants having enhanced ASR resistance comprise detecting in the genome of a plant, presence of (i) one or more favorable SNP markers located at or between positions 152,261 and 345,059 of SEQ ID NO: 1; (ii) one or more favorable SNP markers located at or between positions 272,226 and 297,561 of SEQ ID NO: 1; (iii) one or more favorable SNP markers located at or between positions 278,453 and 290,245 of SEQ ID NO: 1; (iv) one or more markers associated ASR resistance that are within 20 cM, 10 cM, 5 cM, 4 cM, 3 cM, 2 cM, 1 cM, 0.5 cM or 0.1 cM of a SNP marker located at or between positions 152,261 and 345,059 of SEQ ID NO: 1; and/or (v) one or more markers associated ASR resistance that are within 20 cM, 10 cM, 5 cM, 4 cM, 3 cM, 2 cM, 1 cM, 0.5 cM or 0.1 cM of a SNP marker located at or between positions 272,226 and 297,561 of SEQ ID NO: 1.

Methods for producing a disease resistant soybean plant may comprise, consist essentially of or consist of detecting, in a germplasm, a marker associated with enhanced disease resistance (e.g., ASR) wherein said marker is selected from Table 1 or wherein the marker is a closely linked loci of any marker described in Table 1 and producing a soybean plant from said germplasm. The marker may be detected in any sample taken from the germplasm, including, but not limited to, a portion of said germplasm (e.g., a seed chip or leaf punch or a cell from said germplasm) or a nucleotide sequence from said germplasm. Such a sample may be taken from the germplasm using any present or future method known in the art, including, but not limited to, automated methods of removing a portion of endosperm with a sharp blade, drilling a small hole in the seed and collecting the resultant powder, cutting the seed with a laser and punching a leaf disk. The germplasm may be of a non-naturally occurring variety of soybean. In some embodiments, the genome of the germplasm is at least about 50%, 55%, 60%, 65%, 70%, 75%, 80%, 85%, 90%, 95%, 97%, 99% or 100% identical to that of an elite variety of soybean. A Disease resistant soybean plant is then produced from the germplasm identified as having the marker associated with enhanced disease resistance (e.g., ASR) according to methods well known in the art for breeding and producing plants from germplasm.

In some embodiments, the marker detected in the germplasm may comprise, consist essentially of or consist of one or more marker alleles located within a chromosomal interval derived from PI446934 or a progeny thereof wherein said chromosomal interval corresponds with nucleotide base 1 to 264966 of SEQ ID NO: 1, or a portion thereof. In embodiments, the chromosomal interval associated with ASR resistance that is detected by the marker spans 20 cM, 15 cM, 10 cM, 5 cM, 1 cM, 0.5 cM from a SNP marker that associates with increased ASR resistance in soybean wherein the SNP marker is selected from the group consisting of any SNP marker displayed in Table 1. In embodiments, the chromosomal interval associated with ASR resistance comprises SEQ ID NO: 1 or a portion thereof flanked by SNP Marker No. 1850 and SNP Marker No. 3656 of Table 1. In other particular embodiments, the chromosomal interval that confers enhanced ASR resistance comprises SEQ ID NO: 1, or a portion thereof, that is flanked by SNP Marker No. 3115 and SNP Marker No. 3347 of Table 1. In still other embodiments, the chromosomal interval comprises SEQ ID NO: 1 or a portion thereof and one or more of the favorable markers of Table 1. In some embodiments, the marker detected in the germplasm may comprise, consist essentially of or consist of one or more marker alleles selected from Table 1.

Methods for producing and/or selecting an Asian soy rust resistant/tolerant soybean plant or germplasm may comprise crossing a first soybean plant or germplasm with a second soybean plant or germplasm, wherein said first soybean plant or germplasm comprises a chromosomal interval comprising SEQ ID NO: 1 or a portion thereof; or a chromosomal interval derived from PI446934, or a progeny thereof, wherein said chromosomal interval corresponds with nucleotide base 1 to 264966 of SEQ ID NO: 1 or a portion thereof; or a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker of Table 1.

In some embodiments, the chromosomal interval of SEQ ID NO: 1, or a portion thereof, may be introduced through a cis-genic approach.

Also provided herein are methods of introgressing an allele associated with enhanced disease or pathogen resistance (e.g., enhanced resistance to ASR, SCN, SDS, RKN, Phytopthora, etc.) into a soybean plant. Such methods for introgressing an allele associated with enhanced disease (e.g. ASR, SCN, SDS, RKN, Phytopthora, etc.) resistance/tolerance into a soybean plant or germplasm may comprise, consist essentially of or consist of crossing a first soybean plant or germplasm comprising said allele (the donor) wherein said allele is selected from any favorable allele listed in Table 1, or a marker closely linked to a marker listed in Table 1, with a second soybean plant or germplasm that lacks said allele (the recurrent parent) and repeatedly backcrossing progeny comprising said allele with the recurrent parent. Progeny comprising said allele may be identified by detecting, in their genomes, the presence of a marker associated with the enhanced disease or pathogen resistance. The marker may be detected in any sample taken from the progeny, including, but not limited to, a portion of said progeny (e.g., a seed chip, a leaf punch disk or a cell from said plant or germplasm) or a nucleotide sequence from said progeny. Such a sample may be taken from the progeny using any present or future method known in the art, including, but not limited to, automated methods of removing a portion of endosperm with a sharp blade, drilling a small hole in the seed and collecting the resultant powder, cutting the seed with a laser and punching a leaf disk. Either the donor or the recurrent parent, or both, may be of a non-naturally occurring variety of soybean. In some embodiments, the recurrent parent is of an elite variety of soybean. In some embodiments, the genome of the recurrent parent is at least about 50%, 55%, 60%, 65%, 70%, 75%, 80%, 85%, 90%, 95%, 97%, 99% or 100% identical to that of an elite variety of soybean.

In some embodiments, the marker used to identify progeny comprising an allele associated with enhanced disease (e.g., ASR, SCN, SDS, RKN, Phytopthora, brown stem rot etc.) resistance may comprise, consist essentially of or consist of one or more marker alleles located within the chromosomal interval of SEQ ID NO: 1, or a portion thereof; or a chromosomal interval derived from PI446934 or a progeny thereof wherein said chromosomal interval corresponds with nucleotide base 1 to 264966 of SEQ ID NO: 1, or a portion thereof; or a chromosomal interval spanning 20 cM, 15 cM, 10 cM, 5 cM, 1 cM, 0.5 cM from a SNP marker that associates with increased ASR resistance in soybean wherein the SNP marker is any SNP marker of Table 1.

In some embodiments, the marker may comprise, consist essentially of or consist of marker alleles located in at least two different locations of the chromosomal interval of SEQ ID NO: 1, or a portion thereof. For example, the marker may comprise one or more alleles located in the chromosomal interval defined by and including any two markers in Table 1. In example embodiments, the marker may comprise one or more alleles located at or between positions 1 and ??? of SEQ ID NO: 1; one or more alleles located between positions 152,261 and 345,059 of SEQ ID NO: 1; or one or more alleles located at or between positions 272,226 and 297,561 of SEQ ID NO: 1; and/or a combination thereof, such as a first allele located at or between positions 152,261 and 345,059 of SEQ ID NO: 1 and a second allele located at or between positions 272,226 and 297,561 of SEQ ID NO: 1.

In some embodiments, a method for producing a Glycine max plant having increased resistance to ASR is provided, the method comprising the steps of:
a. Providing a Glycine canescens plant line, or progeny thereof comprising a chromosomal interval corresponding to SEQ ID NO: 1, or a portion thereof;
b. Carrying out embryo rescue (as described in U.S. Pat. No. 7,842,850 or transgenically);
c. Collecting the seeds resulting from the method of b)
d. Regenerating the seeds of c) into plants.

In some embodiments, the Glycine canescens plant line of a) is PI446934, or a progeny thereof.

In still further embodiments, a method of identifying or selecting a soybean plant having a ASR resistance allele derived from Glycine canescens is provided, the method comprising the steps of:
a. Isolating a nucleic acid from a soybean plant;
b. Detecting in the nucleic acid the presence of a molecular marker that associates with increased ASR resistance wherein the molecular marker is any marker provided in Table 1, or wherein the molecular marker is a marker located within 20 cM, 10 cM, 5 cM, 1 cM, or 0.5 cM of any marker provided in Table 1; and
c. thereby identifying or selecting a soybean plant having a ASR resistance allele derived from Glycine canescens.

### Disease Resistant Soybean Plants and Germplasms

The present invention provides Disease resistant soybean plants and germplasms. As discussed above, the methods of the present invention may be utilized to identify, produce and/or select a disease resistant soybean plant or germplasm (for example a soybean plant resistant or having increased tolerance to Asian Soybean Rust). In addition to the methods described above, a disease resistant soybean plant or germplasm may be produced by any method whereby a marker associated with enhanced Disease tolerance is introduced into the soybean plant or germplasm, including, but not limited to, transformation, protoplast transformation or fusion, a double haploid technique, embryo rescue, gene editing and/or by any other nucleic acid transfer system.

In some embodiments, the soybean plant or germplasm comprises a non-naturally occurring variety of soybean. In some embodiments, the soybean plant or germplasm is at least about 50%, 55%, 60%, 65%, 70%, 75%, 80%, 85%, 90%, 95%, 97%, 99% or 100% identical to that of an elite variety of soybean.

The disease resistant soybean plant or germplasm may be the progeny of a cross between an elite variety of soybean and a variety of soybean that comprises an allele associated with enhanced Disease tolerance (e.g., ASR) wherein the allele is within a chromosomal interval comprising SEQ ID No: 1, or a portion thereof, or a chromosomal interval derived from PI446934, or a progeny thereof wherein said chromosomal interval corresponds with nucleotide base 1 to 264966 of SEQ ID NO: 1 or a portion thereof; or a chromosomal interval comprising at least one favorable SNP marker of Table 1; or a chromosomal interval spanning 20 cM, 15 cM, 10 cM, 5 cM, 1 cM, 0.5 cM from a SNP marker that associates with increased ASR resistance in soybean wherein the SNP marker is any SNP marker of Table 1.

The Disease resistant soybean plant or germplasm may be the progeny of an introgression wherein the recurrent parent is an elite variety of soybean and the donor comprises an allele associated with enhanced Disease tolerance and/or resistance wherein the donor carries a chromosomal interval comprising SEQ ID NO: 1 or a portion thereof and wherein the chromosome interval comprises at least one favorable allele selected from Table 1.

The Disease resistant soybean plant or germplasm may be the progeny of a cross between a first elite variety of soybean (e.g., a tester line) and the progeny of a cross between a second elite variety of soybean (e.g., a recurrent parent) and a variety of soybean that comprises an allele associated with enhanced ASR tolerance (e.g., a donor).

The Disease resistant soybean plant or germplasm may be the progeny of a cross between a first elite variety of soybean and the progeny of an introgression wherein the recurrent parent is a second elite variety of soybean and the donor comprises an allele associated with enhanced ASR tolerance.

A Disease resistant soybean plant and germplasm of the present invention may comprise one or more markers of the present invention (e.g., one or more of the markers described in Table 1; or any marker in close proximity thereto).

In some embodiments, the Disease resistant soybean plant or germplasm may comprise within its genome, a marker associated with enhanced ASR tolerance, wherein said marker is located within the chromosomal interval of SEQ ID NO: 1; or is a SNP marker of Table 1; or is a marker that lies within a chromosomal interval spanning 20 cM, 15 cM, 10 cM, 5 cM, 1 cM, 0.5 cM from a SNP marker that associates with increased ASR resistance in soybean wherein the SNP marker is selected from the group of SNP markers displayed in Table 1.

In some embodiments, the Disease resistant soybean plant or germplasm may comprise within its genome a marker that comprises, consists essentially of or consists of marker alleles located in at least two different chromosomal intervals. For example, the marker may comprise one or more alleles located in the chromosomal interval defined by and including any combination of two markers of Table 1.

In some embodiments, the disease resistant plant or germplasm is an elite Glycine max plant having introduced into its genome SEQ ID NO: 1, or a portion thereof, and exhibiting increased Asian soy rust (ASR) resistance as compared to a control plant. As non-limiting examples, the genome may comprise one of at least 10%, at least 11%, at least 12%, at least 13%, at least 14%, at least 15%, at least 16%, at least 17%, at least 18%, at least 19%, and at least 20%, at least 21%, at least 22%, at least 23%, at least 24%, at least 25%, at least 26%, at least 27%, at least 28%, at least 29%, and at least 30% of SEQ ID NO: 1. In some embodiments, SEQ ID NO: 1, or the portion thereof, includes a chromosomal interval from Glycine canescens accession line PI446934, or a progeny thereof, and wherein the control plant does not comprise SEQ ID NO: 1, or a portion thereof in its genome. In some embodiments, SEQ ID NO: 1, or a portion thereof, is obtained from Glycine canescens through the use of chromosome doubling, as is known in the art. In some embodiments, SEQ ID NO: 1, or a portion thereof, comprises a SNP marker associated with increased ASR resistance, wherein said SNP marker corresponds with any one of the favorable SNP markers as listed in Table 1. In some embodiments, SEQ ID NO: 1, or a portion of either, corresponds to a position within the Glycine canescens genome and is derived from Glycine canescens chromosome 1, 2, 3, 4, 5, 6, 7, 8, 9, 10, 11, 12, 13, 14, 15, 16, 17, 18, 19, or 20. In one example embodiment, SEQ ID NO: 1, or the portion thereof, corresponds to a position within the Glycine canescens genome and is derived from Glycine canescens chromosome 3. In some embodiments, the plant further shows resistance to any one of the stresses selected from: diseases (such as powdery mildew, Pythium ultimum, Phytophthora root rot, leaf spot, blast, brown spot, root-knot nematode, soybean cyst nematode, soybean vein necrosis virus, soybean stem canker, soybean sudden death syndrome, leaf and neck blast, rust, frogeye leaf spot, brown stem rot, Fusarium, or sheath blight); insect pests (such as whitefly, aphid, grey field slug, sugarcane borer, green bug, or aphid); and abiotic stress (such as drought tolerance, flooding, high level of salinity, heavy metal, aluminum, manganese, cadmium, zinc, UV-B, boron, iron deficiency chlorosis or cold tolerance (i.e. extreme temperatures)).

In some embodiments, the disease resistant plant is a plant of the species Glycine max, wherein a portion of a genome of the plant is obtained from a wild glycine species through the use of one of:
a) chemically induced chromosome doubling; and
b) introgression of a chromosomal interval comprising SEQ ID NO: 1, or a portion thereof.

### Disease Resistant Soybean Seeds

The present invention provides Disease resistant soybean seeds. As discussed above, the methods of the present invention may be utilized to identify, produce and/or select a Disease resistant soybean seed. In addition to the methods described above, a Disease resistant soybean seed may be produced by any method whereby a marker associated with enhanced ASR tolerance is introduced into the soybean seed, including, but not limited to, transformation, protoplast transformation or fusion, a double haploid technique, embryo rescue, genetic editing (e.g., CRISPR or TALEN or MegaNucleases) and/or by any other nucleic acid transfer system.

In some embodiments, the Disease resistant soybean seed comprises a non-naturally occurring variety of soybean. In some embodiments, the soybean seed is at least about 50%, 55%, 60%, 65%, 70%, 75%, 80%, 85%, 90%, 95%, 97%, 99% or 100% identical to that of an elite variety of soybean.

The Disease resistant soybean seed may be produced by a Disease resistant soybean plant identified, produced or selected by the methods of the present invention. In some embodiments, the Disease resistant soybean seed is produced by a Disease resistant soybean plant of the present invention.

A disease resistant soybean seed of the present invention may comprise within its genome SEQ ID NO: 1, or a portion thereof, wherein a plant produced by growing the seed exhibits increased Asian soy rust (ASR) resistance. As non-limiting examples, the genome of the seed may comprise one of at least 10%, at least 11%, at least 12%, at least 13%, at least 14%, at least 15%, at least 16%, at least 17%, at least 18%, at least 19%, and at least 20%, at least 21%, at least 22%, at least 23%, at least 24%, at least 25%, at least 26%, at least 27%, at least 28%, at least 29%, and at least 30% of SEQ ID NO: 1.

A disease resistant soybean seed of the present invention may comprise one or more markers from Table 1 of the present invention.

In some embodiments, the Disease resistant soybean seed may comprise within its genome, a marker associated with enhanced ASR tolerance, wherein said marker is located within a chromosomal interval comprising SEQ ID NO: 1, or a portion thereof. In embodiments, the marker is any SNP marker of Table 1 that associates with increased ASR resistance in soybean.

### NON-LIMITING EMBODIMENTS

Non-limiting embodiments include:

Non-limiting embodiments of plants having increased disease resistance:
1. An elite Glycine max plant having introgressed into its genome a chromosomal interval comprising the nucleotide sequence of SEQ ID NO: 1, or a portion thereof, wherein the plant exhibits increased resistance to Asian Soy Rust (ASR) as compared to a control plant not comprising the chromosomal interval or portion thereof.
2. The plant of embodiment 1, wherein the portion of the chromosomal interval of SEQ ID NO: 1 comprises at least 10%; at least 11%; at least 12%; at least 13%; at least 14%; at least 15%; at least 16%%; at least 17%; at least 18%; at least 19%; at least 20%; at least 21%; at least 22%; at least 23%; at least 24%; at least 25%; at least 26%; at least 27%; at least 28%; at least 29%; at least 30%; at least 31%; at least 32%; at least 33%; at least 34%; at least 35%; at least 36%; at least 37%; at least 38%; at least 39%; at least 40%; at least 41%; at least 42%; at least 43%; at least 44%; at least 45%; at least 46%; at least %; at least 48%; at least 49%; or at least 50%; at least 51%; at least 52%; at least 53%; at least 54%; at least 55%; at least 56%; at least 57%; at least 58%; at least 59%; or at least 60%; at least 61%; at least 62%; at least 63%; at least 64%; at least 65%; at least 66%; at least 67%; at least 68%; at least 69%; at least 70%; at least 71%; at least 72%; at least 73%; at least 74%; at least 75%; at least 76%; at least 77%; at least 78%; at least 79%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; or at least 99% of SEQ ID NO: 1.
3. The plant of any one of embodiments 1-2, wherein the chromosomal interval of SEQ ID NO: 1, or the portion thereof, is from the genome of Glycine canescens accession line PI446934, or a progeny thereof.
4. The plant of any of embodiments 1-3, wherein the chromosomal interval comprising SEQ ID NO: 1 or a portion thereof comprises a SNP marker associated with increased ASR resistance, wherein said SNP marker corresponds with any one of the favorable SNP markers as listed in Table 1.
5. The plant of any of embodiments 1-5, wherein the nucleotide sequence comprising SEQ ID NO: 1 or the portion of either corresponds to a position within the Glycine canescens genome and is derived from Glycine canescens chromosome 1, 2, 3, 4, 5, 6, 7, 8, 9, 10, 11, 12, 13, 14, 15, 16, 17, 18, 19, or 20.
6. The plant of embodiment 6, wherein the nucleotide sequence comprising SEQ ID NO: 1 or the portion of either is derived from Glycine canescens chromosome 3.
7. A progeny plant or seed of any of embodiments 1-6.
8. A plant cell or plant part derived from the plant of any of embodiments 1-7.
9. The plant of any one of embodiments 1-8, wherein the plant further shows resistance to any one of the stresses selected from:
   (a) diseases including powdery mildew, Pythium ultimum, Phytophthora root rot, leaf spot, blast, brown spot, root-knot nematode, soybean cyst nematode, soybean vein necrosis virus, soybean stem canker, soybean sudden death syndrome, leaf and neck blast, rust, frogeye leaf spot, brown stem rot, Fusarium, or sheath blight;
   (b) insect pests including whitefly, aphid, grey field slug, sugarcane borer, green bug, or aphid;
   (c) abiotic stress including drought tolerance, flooding, high level of salinity, heavy metal, aluminum, manganese, cadmium, zinc, UV-B, boron, iron deficiency chlorosis or cold tolerance.
10. An elite soybean plant comprising an ASR resistance allele which confers the plant with increased resistance to ASR, and wherein the ASR allele comprises at least one single nucleotide polymorphism (SNP) selected from the group of “favorable” SNPs described in any one of Table 1.
11. An elite soybean plant comprising a chromosomal interval derived from Glycine canescens and comprising at least one favorable SNP marker selected from any one of Table 1.
12. The plant of embodiment 10, wherein the Glycine canescens is accession line PI446934 or a progeny thereof.
13. An elite Glycine max plant having introduced into its genome SEQ ID NO: 1 or a portion thereof, and exhibiting increased Asian soy rust (ASR) resistance as compared to a control plant, wherein SEQ ID NO: 1, or a portion thereof is introduced into its genome through the use of introgression of a chromosomal interval comprising SEQ ID NO: 1, or a portion of either, from a wild glycine species and/or chemically induced chromosome doubling.
14. The elite Glycine max plant of embodiment 13, wherein the chromosomal interval is introduced from a Glycine canescens plant line.
15. The elite Glycine max plant of embodiment 14, wherein the Glycine canescens plant line is accession line PI446934, or a progeny thereof.
16. The elite Glycine max plant of any of embodiments 13-15, wherein the chromosomal interval is introduced from chromosome 1, 2, 3, 4, 5, 6, 7, 8, 9, 10, 11, 12, 13, 14, 15, 16, 17, 18, 19, or 20 of Glycine canescens.
17. The elite Glycine max plant of embodiment 16, wherein the chromosomal interval is introduced from chromosome 3 of Glycine canescens.
18. The elite Glycine max plant of any of embodiments 13-17 wherein SEQ ID NO: 1, or a portion thereof, comprises a SNP marker associated with increased ASR resistance, wherein said SNP marker corresponds with any one of the favorable SNP markers as listed in Table 1.
19. The elite Glycine max plant of any of embodiments 17-22, wherein the chromosomal interval comprises at least 10%; at least 11%; at least 12%; at least 13%; at least 14%; at least 15%; at least 16%%; at least 17%; at least 18%; at least 19%; at least 20%; at least 21%; at least 22%; at least 23%; at least 24%; at least 25%; at least 26%; at least 27%; at least 28%; at least 29%; at least 30%; at least 31%; at least 32%; at least 33%; at least 34%; at least 35%; at least 36%; at least 37%; at least 38%; at least 39%; at least 40%; at least 41%; at least 42%; at least 43%; at least 44%; at least 45%; at least 46%; at least 47%; at least 48%; at least 49%; or at least 50%; at least 51%; at least 52%; at least 53%; at least 54%; at least 55%; at least 56%; at least 57%; at least 58%; at least 59%; or at least 60%; at least 61%; at least 62%; at least 63%; at least 64%; at least 65%; at least 66%; at least 67%; at least 68%; at least 69%; at least 70%; at least 71%; at least 72%; at least 73%; at least 74%; at least 75%; at least 76%; at least 77%; at least 78%; at least 79%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; or at least 99% of SEQ ID NO: 1.
20. A progeny plant or seed of any of embodiments 13-19.
21. A plant cell or plant part derived from the plant of any of embodiments 13-20.

Non-limiting embodiments of methods for producing, selecting, and/or detecting plants having increased disease resistance:
22. A method for producing a Glycine max plant having increased resistance to ASR, the method comprising the steps of:
   a. Providing a Glycine canescens plant line, or progeny thereof comprising a chromosomal interval corresponding to SEQ ID NO: 1;
   b. Carrying out the embryo rescue method essentially as described in Example 4 or as described in U.S. Pat. No. 7,842,850, or transgenically;
   c. Collecting the seeds resulting from the method of b)
   d. Regenerating the seeds of c) into plants.
23. The method of embodiment 22, wherein the Glycine canescens plant line of a) is accession line PI446934 or a progeny thereof.
24. A method of identifying or selecting a soybean plant having a ASR resistance allele derived from Glycine canescens, the method comprising the steps of:
   a. Isolating a nucleic acid from a soybean plant;
   b. Detecting in the nucleic acid the presence of a molecular marker that associates with increased ASR resistance wherein the molecular marker is located within 20 cM, 10 cM, 5 cM, 1 cM, 0.5 cM of a marker as described in Table 1; and
   c. Thereby identifying or selecting a soybean plant having a ASR resistance allele derived from Glycine canescens.

### EXAMPLES

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1 Identification of ASR Resistant Wild Glycine Line

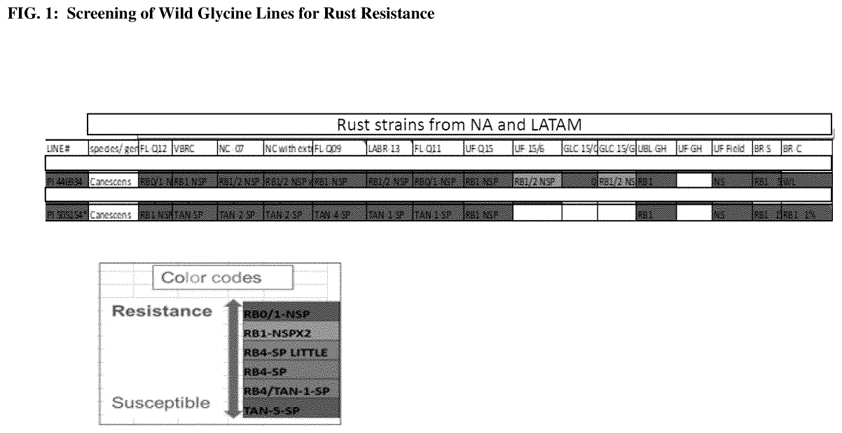

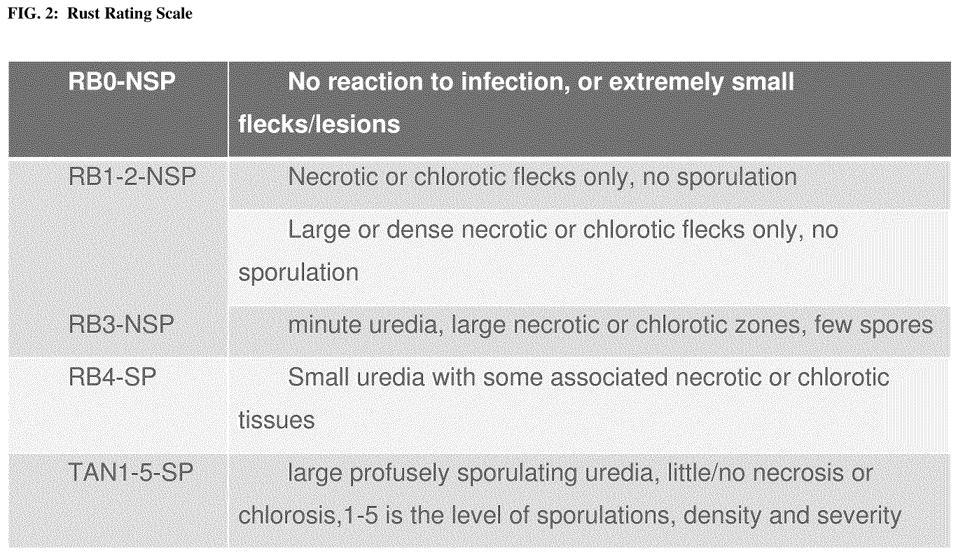

Wild glycine lines, including G. canescens PI446934 and PI505154, were evaluated for rust resistance against sixteen rust strains collected across a diverse range of environments (FIG. 1). The rust data were generated using single pustule derived isolates from USDA-ARS (FL Q09, FL Q12, LABR13, FLQ11) and field populations (FL Q15, NC06, Vero, GLC15, UBL, BR south and BR central), the screening was carried out in contained facilities (FL Q09, FL Q12, LABR13, FLQ11, FL Q15, NC06, Vero, GLC15, UBL, BR South, BR central). The wild glycine lines were evaluated over a multiple day course of infection and rated at various time points. The rating and evaluation were performed using methods well known in the art, based upon Burdon and Speer (Euphytica, 33: 891-896, 1984; also TAG, 1984). An example rating table is shown in FIG. 2. The accession lines were screened >2 times with ˜4 plants each time in North & South America using the large diverse panel of rust isolates.

### Example 2 Allele Mining & Associations to PI446934 ASR Loci

The resistant parent was crossed to a susceptible G. canescens line and an F1 plant was generated (See Table 2). The F1 plant was self-fertilized and F2 seed was harvested from the selfed F1 plant. Around 200 F2 seed were sown and leaf tissue from each plant was collected for DNA preparations and then the plants were inoculated with Phakopsora pachyrhizi to determine the resistance/susceptible phenotype of each F2 individual. Tissue from 50 resistant F2s and 50 susceptible F2s were combined in separate pools and genomic DNA was prepared from each pool. Illumina sequencing libraries were prepared from DNA for each of the pools and each library was sequenced in two Illumina HiSeq2000 2×100 bp Paired-End (PE) lanes. The average yield per sample was 383 million read pairs, which equals 77 gigabases of sequence per library. The sequencing reads were trimmed to remove bases with PHRED quality scores of <15.

Quality trimmed reads were aligned to an internal PI446934 reference genome sequence using GSNAP (WU and NACU 2010) as paired-end fragments. If a pair of reads could not be aligned together, they were treated as singletons for alignment. Reads were used in subsequent analyses if they mapped uniquely to the reference (≤2 mismatches every 36 bp and less than 5 bases for every 75 bp as tails).

SNPs were filtered prior to BSA analysis based on read depth, with SNPs having between 40 and 200× read depth being retained. A Chi-square test was used to select SNPs with significantly different read counts between the two alleles in the two pools. An empirical Bayesian approach (LIU et al. 2012) was used to estimate the conditional probability that there is no recombination between each SNP marker and the causal locus in both the resistant pool and in the susceptible pool. The probability of the linkage between the SNP and the causal gene is the geometric mean of these two conditional probabilities. Around 1000 SNPs were found to have possible linkage to the target locus. A subset of these putatively linked SNPs was used to fine map the locus using phenotyped F2 individuals (Liu, S., C.-T. Yeh, H. M. Tang, D. Nettleton and P. S. Schnable, 2012 Gene Mapping via Bulked Segregant RNA-Seq (BSR-Seq). PLoS ONE 7: e36406 & Wu, T. D., and S. Nacu, 2010 Fast and SNP-tolerant detection of complex variants and splicing in short reads. Bioinformatics 26: 873-881).

TABLE 2
Plant Crossings & Study Type

| | | | | F2: Resistant |
|---|---|---|---|---|
| | PI# (male, | PI # (female, | Study | to Susceptible |
| Species | resistant) | susceptible) | Type | Ratio |
| G. canescens | PI 446934 | PI 505154 | BSA | 3:01 |
| G. canescens | PI 446934 | PI 505154 | BSR + DEG | 3:01 |
| G. canescens | PI 446934 | PI 505154 | Indexing | 3:01 |

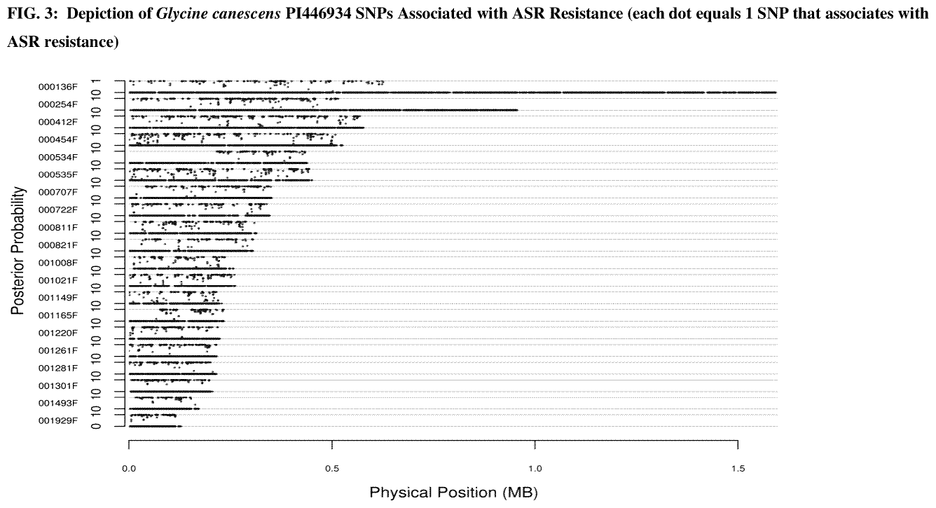

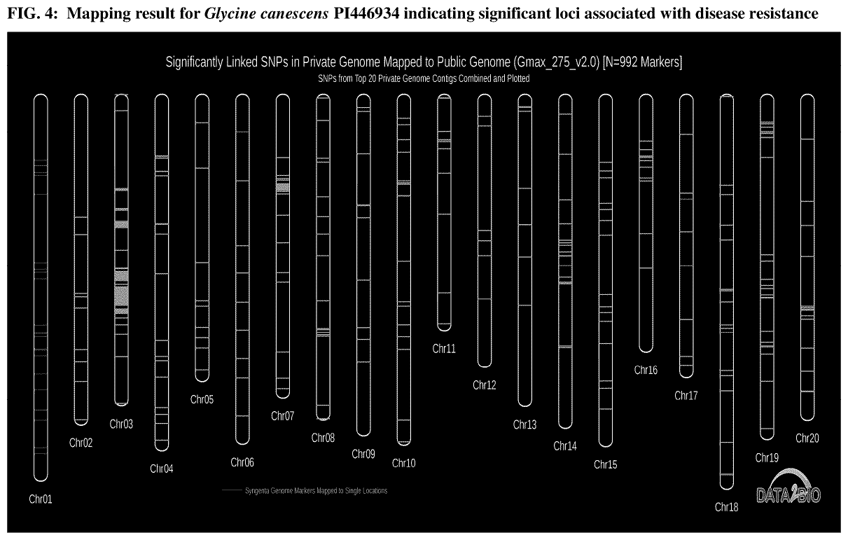

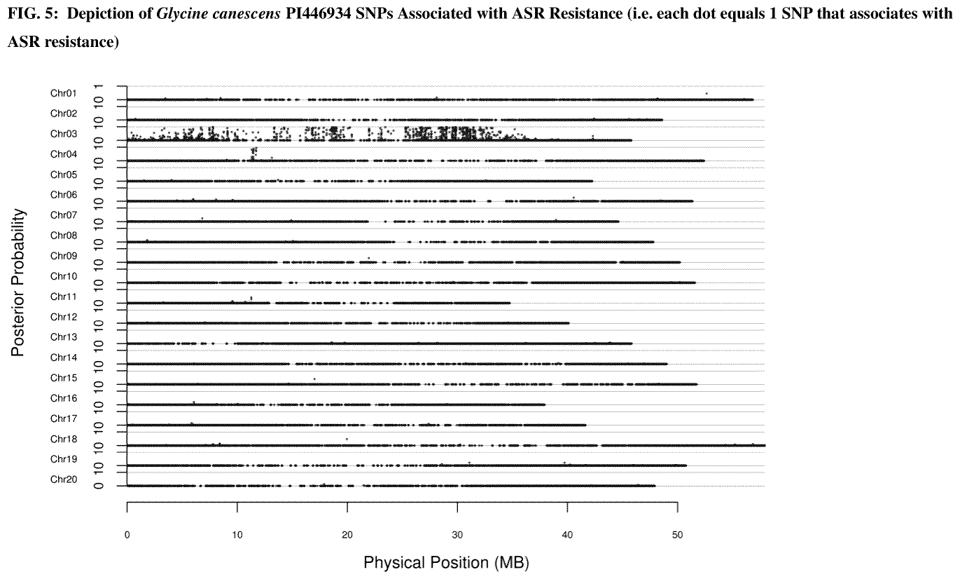

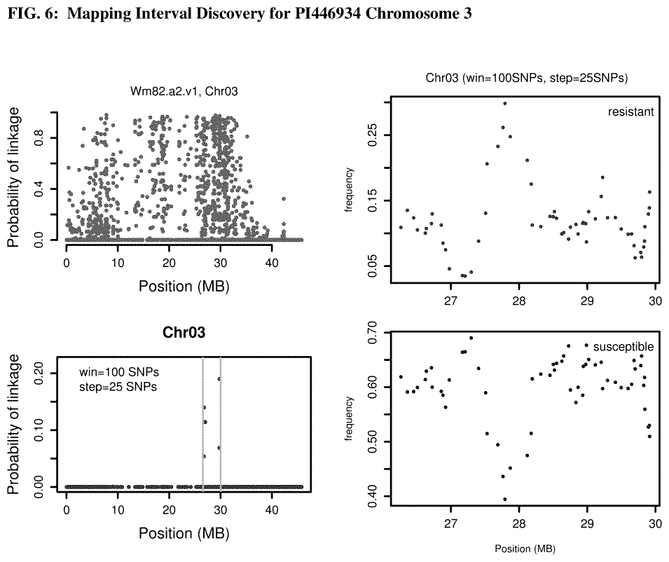

Chromosome discovery for causal loci in the tetraploid soybean population, PI446934 was carried out using Data2Bio's Genomic Bulked Segregant Analysis (gBSA) technology. Data2Bio generated two libraries from RNA samples extracted from one susceptible tissue pool and one resistant tissue pool. After various filtering steps, informative SNPs were identified based on the internal PI446934 genome and Williams 82 public genome. A Bayesian approach was then used to calculate trait-associated probabilities. Next, a physical map of trait-associated SNPs on the identified contigs was created. One contig, Scaffold 000454F (SEQ ID NO: 1), showed a high density of SNPs associated with ASR resistance, as shown in FIGS. 3-6. The context sequences associated with these SNPs were also aligned to the publicly available G. max genome (Williams 82 v2.0 from soybase(.)org) to create a chromosome-level understanding of the mapping interval. The chromosomal positions of the trait-associated SNPs were then displayed graphically. Most of the SNPs from the mapping interval clustered on a small region of chromosome 3 (see FIGS. 3-6).

### Example 3: Fine-Mapping of an ASR Resistance QTL on Chromosome 3

F1 lines from pedigree PI505154/PI446934 were selected, self-crossed, and harvested. Recombinants at the F2 generation from pedigree PI505154/PI446934 were screened using a set of 150 TaqMan assays targeted to the region of interest on as well as surrounding regions on Chromosome 3. Those plants showing recombinations in the region of interest based on the subset of 41 SNP assays (Tables 3 and 4) with good segregation patterns were phenotyped using rust isolates and their rust reaction compared with expectations based on their inferred parental haplotypes. Those plants passing this test were then sequenced using ˜5× Illumina short reads to get finer detail on their recombination location. Also, F2:3 selfed progeny of each sequenced F2 plant were screened for rust reaction, to allow determination of rust reaction as homozygous resistant, segregating, or homozygous susceptible.

In particular, given the sequence of the reference genome and the SNP allele associated with ASR resistance, oligonucleotide primers were developed and used to assay for the SNP. In an exemplary two-step assay, a forward primer, a reverse primer, and two assay probes (or hybridization oligos) are employed. The forward and reverse primers are employed to amplify genetic loci that comprise SNPs that are associated with ASR resistance loci. The particular nucleotides that are present at the SNP positions are then assayed using the assay primers, which in example embodiments are differentially labeled with fluorophores to permit distinction between the two assay probes in a single reaction.

Table 3 provides a list of example assay IDs, wherein each assay ID corresponds to a particular SNP position within the chromosomal interval represented by SEQ ID NO: 1. The assays are designed to differentiate between favorable and unfavorable alleles associated with a given SNP position, as indicated.

Table 4 provides a list and sequence of the assay components used in each of the assays listed in Table 3. Particularly, Table 4 lists the target sequence amplified to identify the SNP, sequences of the specific forward and reverse primers, as well as the sequence and combination of fluorophores used for each of the assays. In the listing of the assay components, the assay component ID indicates the associated assay ID (Table 3) and the nature of the component (whether it is a probe or a primer). The suffix F1 indicates that the corresponding sequence is for a forward primer, the suffix R1 indicates that the corresponding sequence is for a reverse primer, the suffix FM indicates that the corresponding sequence is for an assay probe having the FAM fluorophore, and the suffix TT indicates that the corresponding sequence is for an assay probe having the TET fluorophore. For example, “S2109FM”, “S2109TT”, “S2109F1” and “S2109R1” refer, respectively, to the FAM probe, TET probe, forward primer, and reverse primer for Assay ID S2109 used for identification of the allele corresponding to SNP ID No. 3006 of Table 1 and 3, which is the SNP at position 266523 of SEQ ID NO: 1. The target sequence amplified is SEQ ID NO: XX.

One of skill in the art will recognize that sequences to either side of the given primers can be used in place of the given primers, so long as the primers can amplify a region that includes the allele to be detected. The precise probe used for detection can vary, e.g., any probe that can identify the region of a marker amplicon to be detected can be substituted for those probes exemplified herein. Configuration of the amplification primers and detection probes can also be varied. Thus, the invention is not limited to the primers, probes, or marker sequences specifically recited herein.

TABLE 3
Assays associated with SNP positions within SEQ ID NO:
1 that are associated with increased resistance to ASR

| Position | | | Favorable | Unfavorable |
|---|---|---|---|---|
| within SEQ | SNP ID in | | Allele (Rust | allele (Rust |
| ID NO: 1 | Table 1 | Assay ID | Resistant) | Susceptible) |
| 38036 | | S21188 | T | C |
| 38603 | | S21231 | G | C |
| 43041 | | S21220 | A | G |
| 68515 | | S21217 | T | G |
| 69709 | | S21243 | A | G |
| 111190 | | S2144 | A | G |
| 152839 | 1856 | S21196 | T | C |
| 153137 | 1859 | S21242 | A | G |
| 163395 | 1909 | S21205 | G | A |
| 259760 | 2912 | S21210 | C | T |
| 260225 | 2916 | S21232 | G | A |
| 261570 | 2941 | S21239 | G | A |
| 263015 | 2957 | S21212 | G | A |
| 264592 | 2982 | S21206 | A | C |
| 265945 | 2992 | S21228 | C | T |
| 266523 | 3006 | S2109 | C | T |
| 272005 | 3111 | S21215 | A | G |
| 273806 | 3145 | S21237 | C | T |
| 277008 | 3204 | S2187 | A | T |
| 292971 | 3332 | S21198 | A | C |
| 296832 | 3344 | S21208 | C | A |
| 307547 | 3375 | S21181 | A | T |
| 308028 | 3382 | S21184 | G | T |
| 310922 | 3413 | S21227 | C | A |
| 314305 | 3434 | S21229 | G | C |
| 319546 | 3461 | S21234 | G | A |
| 322998 | 3476 | S21178 | T | G |
| 350762 | | S21194 | C | A |
| 351819 | | S21187 | T | C |
| 352378 | | S21219 | A | G |
| 356512 | | S21195 | T | A |
| 356803 | | S21185 | G | C |
| 365615 | | S21233 | T | A |
| 370711 | | S21235 | G | A |
| 376950 | | S21244 | T | C |
| 377789 | | S21197 | A | G |
| 389946 | | S256 | T | C |
| 415434 | | S21213 | A | G |
| 416589 | | S21238 | G | A |
| 418116 | | S21214 | T | C |
| 493279 | | S21191 | A | G |
| 495329 | | S21203 | G | A |
| 503769 | | S21204 | C | A |
| 507251 | | S21224 | G | A |
| 508264 | | S21189 | G | T |

TABLE 4
Sequences of assay components used in assays associated with SNP positions
within SEQ ID NO: 1. Suffix “F1” refers to a forward primer; Suffix “R1” refers to a
reverse primer. Primers with a common prefix can form a primer pair. Suffix FM and TT
refer to probes.

| | | | | Assay | | |
|---|---|---|---|---|---|---|
| | Assay | Assay | Assay | component | | |
| Assay | Target | Component | Component | SEQ ID | | |
| ID | Sequence | Name | Sequence | NO: | Flurophore | |
| S2109 | SEQ ID NO: | S2109A1FM | TTAACGTTCCAA | 3 | FAM | A |
| | 2 | | AAACC | | | |
| S2109 | | S2109A2TT | TAACGTTCCAGA | 4 | TET | G |
| | | | AACC | | | |
| S2109 | | S2109F1 | GACACCTAGCCC | 5 | | |
| | | | ATGTCAAA | | | |
| S2109 | | S2109R1 | CATGTTGGTTCA | 6 | | |
| | | | GATTCCTATTTA | | | |
| | | | TTCC | | | |
| S21178 | SEQ ID NO: | S21178A1FM | ACGTCCCTCGAA | 8 | FAM | C |
| | 7 | | CCAG | | | |
| S21178 | | S21178A2TT | AAACGTCCCTCT | 9 | TET | A |
| | | | AACCAGT | | | |
| S21178 | | S21178F1 | TGATGAATGGC | 10 | | |
| | | | AGTGTGAAACCT | | | |
| S21178 | | S21178R1 | ACCAGAGAGTTT | 11 | | |
| | | | AGAGCTGCATA | | | |
| S21181 | SEQ ID NO: | S21181A1FM | TGCCATAAATTG | 13 | FAM | T |
| | 12 | | TCATTTAAA | | | |
| S21181 | | S21181A2TT | TGCCATAAATTG | 14 | TET | A |
| | | | TCTTTTAAA | | | |
| S21181 | | S21181F1 | TTTCTGGTCCCT | 15 | | |
| | | | ACCATTAGGTTA | | | |
| S21181 | | S21181R1 | TGTTGGTGTGCC | 16 | | |
| | | | TCAATGAAG | | | |
| S21184 | SEQ ID NO: | S21184A1FM | TTCTGAAGTTTT | 18 | FAM | C |
| | 17 | | GTGGGAAC | | | |
| S21184 | | S21184A2TT | CTGAAGTTTTGT | 19 | TET | A |
| | | | TGGAACAGG | | | |
| S21184 | | S21184F1 | CAAGCACTCCCG | 20 | | |
| | | | TTGGAGATG | | | |
| S21184 | | S21184R1 | GGAAAGACGAA | 21 | | |
| | | | GAGTTTGAACA | | | |
| | | | GA | | | |
| S21185 | SEQ ID NO: | S21185A1FM | TGACTGAAATTA | 23 | FAM | G |
| | 22 | | TCCTACCTT | | | |
| S21185 | | S21185A2TT | TGACTGAAATTA | 24 | TET | C |
| | | | TCGTACCTT | | | |
| S21185 | | S21185F1 | TCCAATTATCCA | 25 | | |
| | | | GTGATCTCCGA | | | |
| S21185 | | S21185R1 | TTATGGCTATCA | 26 | | |
| | | | GACCACTTCCC | | | |
| S21187 | SEQ ID NO: | S21187A1FM | AGAGCAGGATC | 28 | FAM | G |
| | 27 | | GCTTGAT | | | |
| S21187 | | S21187A2TT | AGAGCAGGATT | 29 | TET | A |
| | | | GCTTGATG | | | |
| S21187 | | S21187F1 | CAGCCATGTTTG | 30 | | |
| | | | GCCTCATTG | | | |
| S21187 | | S21187R1 | TGGTTGTATGGT | 31 | | |
| | | | GGATCTTCTTGG | | | |
| | | | T | | | |
| S21188 | SEQ ID NO: | S21188A1FM | TTAGTGATTGGA | 33 | FAM | A |
| | 32 | | GAACATCTTG | | | |
| S21188 | | S21188A2TT | AGTGATTGGAG | 34 | TET | G |
| | | | AGCATCTTG | | | |
| S21188 | | S21188F1 | GGTGGTGAGGG | 35 | | |
| | | | AAGGTCTTT | | | |
| S21188 | | S21188R1 | GGCGTCTCTTCC | 36 | | |
| | | | GATTTCAAC | | | |
| S21189 | SEQ ID NO: | S21189A1FM | AACTTCTCCTCA | 38 | FAM | A |
| | 37 | | AACCAGA | | | |
| S21189 | | S21189A2TT | TTCTCCTCCAAC | 39 | TET | C |
| | | | CAGA | | | |
| S21189 | | S21189F1 | CCTTTCAATGAC | 40 | | |
| | | | TCTTGTTCTTCC | | | |
| S21189 | | S21189R1 | GACCTGAGAGA | 41 | | |
| | | | GTTGACGGA | | | |
| S21191 | SEQ ID NO: | S21191A1FM | AGGCAATAAAT | 43 | FAM | G |
| | 42 | | ACCATCGATAC | | | |
| S21191 | | S21191A2TT | CAGGCAATAAAT | 44 | TET | A |
| | | | ACCATTGATAC | | | |
| S21191 | | S21191F1 | TGAAACCTTCTC | 45 | | |
| | | | GATACTTCCTTC | | | |
| S21191 | | S21191R1 | GGCTTGTTGCCT | 46 | | |
| | | | TTCATGCTA | | | |
| S21194 | SEQ ID NO: | S21194A1FM | TGATGCTTAGCA | 48 | FAM | C |
| | 47 | | TTGCA | | | |
| S21194 | | S21194A2TT | TGATGCTTATCA | 49 | TET | A |
| | | | TTGCAA | | | |
| S21194 | | S21194F1 | GTTATGAGAGT | 50 | | |
| | | | GAAGCCAAGTG | | | |
| | | | AA | | | |
| S21194 | | S21194R1 | TCAGCAAGTTCT | 51 | | |
| | | | GCCAAGACAT | | | |
| S21195 | SEQ ID NO: | S21195A1FM | CTCGAGTTACTT | 53 | FAM | A |
| | 52 | | AGCGTATG | | | |
| S21195 | | S21195A2TT | TCGAGTTACTTT | 54 | TET | T |
| | | | GCGTATG | | | |
| S21195 | | S21195F1 | CGGAAGAACTTT | 55 | | |
| | | | GCCATCTTAGTG | | | |
| S21195 | | S21195R1 | GCAGTGCTCTGA | 56 | | |
| | | | TTTCAAATTCAA | | | |
| S21196 | SEQ ID NO: | S21196A1FM | TGGTCGGAGAA | 58 | FAM | A |
| | 57 | | GGAAAGT | | | |
| S21196 | | S21196A2TT | TGGTCGGAGGA | 59 | TET | G |
| | | | GGAAAGT | | | |
| S21196 | | S21196F1 | CGGGAAATTCC | 60 | | |
| | | | GGCGAGA | | | |
| S21196 | | S21196R1 | CGCTCCTCACTT | 61 | | |
| | | | CGAGTCTTC | | | |
| S21197 | SEQ ID NO: | S21197A1FM | CTTCAACTTCCG | 63 | FAM | G |
| | 62 | | ATAGGG | | | |
| S21197 | | S21197A2TT | CCACTTCAACTT | 64 | TET | A |
| | | | CTGATAG | | | |
| S21197 | | S21197F1 | GCACGTACCTTC | 65 | | |
| | | | TTCAAGCGAAT | | | |
| S21197 | | S21197R1 | AGCTCAGCGTCG | 66 | | |
| | | | TCGTCTA | | | |
| S21198 | SEQ ID NO: | S21198A1FM | TGTATCTGCTGC | 68 | FAM | C |
| | 67 | | TTCTG | | | |
| S21198 | | S21198A2TT | TGTATCTGCTTC | 69 | TET | A |
| | | | TTCTGT | | | |
| S21198 | | S21198F1 | ACGAGTGAACT | 70 | | |
| | | | GTGCTCCAA | | | |
| S21198 | | S21198R1 | GGACCCAATTCA | 71 | | |
| | | | CTATGGCTTGT | | | |
| S21203 | SEQ ID NO: | S21203A1FM | CCGTATTGGATG | 73 | FAM | A |
| | 72 | | GATTGGT | | | |
| S21203 | | S21203A2TT | ATTGGATGGGTT | 74 | TET | G |
| | | | GGTGAC | | | |
| S21203 | | S21203F1 | CCTGAGACAGTA | 75 | | |
| | | | CATGTGGATGA | | | |
| S21203 | | S21203R1 | TGTGGCCCTGTA | 76 | | |
| | | | CCTCTAGTA | | | |
| S21204 | SEQ ID NO: | S21204A1FM | CATCTGCCTCGA | 78 | FAM | C |
| | 77 | | ACAT | | | |
| S21204 | | S21204A2TT | TGCATCTGCCTC | 79 | TET | A |
| | | | TAACAT | | | |
| S21204 | | S21204F1 | CTCAGCAGCAGC | 80 | | |
| | | | AACCAA | | | |
| S21204 | | S21204R1 | GTGTTCCTTTGA | 81 | | |
| | | | TCTGTGCATCTT | | | |
| | | | A | | | |
| S21205 | SEQ ID NO: | S21205A1FM | TAGAGTCTGTAA | 83 | FAM | A |
| | 82 | | CAATTGCC | | | |
| S21205 | | S21205A2TT | CTAGAGTCTGTA | 84 | TET | G |
| | | | ACGATTGC | | | |
| S21205 | | S21205F1 | GCCCACCTTTAA | 85 | | |
| | | | CTGATCTTGTGA | | | |
| | | | T | | | |
| S21205 | | S21205R1 | GAGACGTACAC | 86 | | |
| | | | AGGAGAGATCT | | | |
| | | | AAT | | | |
| S21206 | SEQ ID NO: | S21206A1FM | CAGCTCATATTC | 88 | FAM | A |
| | 87 | | ATCAATGTA | | | |
| S21206 | | S21206A2TT | AGCTCATATTCA | 89 | TET | C |
| | | | TCCATGTATT | | | |
| S21206 | | S21206F1 | TTCCTGTGCTGT | 90 | | |
| | | | AGGTTGGT | | | |
| S21206 | | S21206R1 | CCGCCACCCATA | 91 | | |
| | | | TAACTGCTA | | | |
| S21208 | SEQ ID NO: | S21208A1FM | ACAAGTTCCGGT | 93 | FAM | C |
| | 92 | | TCCTTAT | | | |
| S21208 | | S21208A2TT | CAAGTTCCGTTT | 94 | TET | A |
| | | | CCTTA | | | |
| S21208 | | S21208F1 | CCGTTCCGTGGA | 95 | | |
| | | | AAGCTTCAG | | | |
| S21208 | | S21208R1 | CCACAATATGGT | 96 | | |
| | | | TAGTTTCCCTCC | | | |
| | | | T | | | |
| S21210 | SEQ ID NO: | S21210A1FM | AAACAAAACCCC | 98 | FAM | A |
| | 97 | | ACAAAGGAG | | | |
| S21210 | | S21210A2TT | ACAAAACCCCAC | 99 | TET | G |
| | | | GAAGGA | | | |
| S21210 | | S21210F1 | GGGATGAGAAA | 100 | | |
| | | | ACCCTAACGAGA | | | |
| | | | A | | | |
| S21210 | | S21210R1 | TCCATTCGGAGG | 101 | | |
| | | | CTCTAGCAT | | | |
| S21212 | SEQ ID NO: | S21212A1FM | ACGGGTTTGTTA | 103 | FAM | A |
| | 102 | | CATTTGG | | | |
| S21212 | | S21212A2TT | CGGGTTTGTTAC | 104 | TET | G |
| | | | GTTTGG | | | |
| S21212 | | S21212F1 | CTGCTGCTCGTA | 105 | | |
| | | | GGAAGGA | | | |
| S21212 | | S21212R1 | CACCCAACTCTG | 106 | | |
| | | | TGCCTGTAAT | | | |
| S21213 | SEQ ID NO: | S21213A1FM | CGTCCATCTCAT | 108 | FAM | A |
| | 107 | | TACG | | | |
| S21213 | | S21213A2TT | CGTCCATCTCGT | 109 | TET | G |
| | | | TACG | | | |
| S21213 | | S21213F1 | TGATGCGTCGTT | 110 | | |
| | | | GACTTGTTTC | | | |
| S21213 | | S21213R1 | TGCGTGCAAATG | 111 | | |
| | | | GTCGTC | | | |
| S21214 | SEQ ID NO: | S21214A1FM | CACACTCACCAC | 113 | FAM | G |
| | 112 | | CTGA | | | |
| S21214 | | S21214A2TT | AGCACACTCATC | 114 | TET | A |
| | | | ACCTG | | | |
| S21214 | | S21214F1 | CCTCCTCTCCAA | 115 | | |
| | | | ATTCATTTCTTCA | | | |
| S21214 | | S21214R1 | CCTTGCTTCAGC | 116 | | |
| | | | TTTCCTTCCATT | | | |
| S21215 | SEQ ID NO: | S21215A1FM | TTTAAATGGAAA | 118 | FAM | A |
| | 117 | | CCCAAACC | | | |
| S21215 | | S21215A2TT | TAAATGGAAGC | 119 | TET | G |
| | | | CCAAACC | | | |
| S21215 | | S21215F1 | GGACACAATAG | 120 | | |
| | | | GCTTGGAAGGT | | | |
| S21215 | | S21215R1 | TGCCAAAGTTGG | 121 | | |
| | | | AGGAATCACC | | | |
| S21217 | SEQ ID NO: | S21217A1FM | CACTCTGTATTT | 123 | FAM | A |
| | 122 | | TACAGTTAATT | | | |
| S21217 | | S21217A2TT | TCTGTATTTTAC | 124 | TET | C |
| | | | CGTTAATTG | | | |
| S21217 | | S21217F1 | AGGCTGGAGGT | 125 | | |
| | | | GCTTACACATTG | | | |
| S21217 | | S21217R1 | TGCTCCGGACAG | 126 | | |
| | | | TGACA | | | |
| S21219 | SEQ ID NO: | S21219A1FM | TAACTTGGTCGC | 128 | FAM | G |
| | 127 | | ATTGTG | | | |
| S21219 | | S21219A2TT | CAGTAACTTGGT | 129 | TET | A |
| | | | TGCATTG | | | |
| S21219 | | S21219F1 | CACAGCTCCTGG | 130 | | |
| | | | TGTTCCAAT | | | |
| S21219 | | S21219R1 | CACTATGATCTC | 131 | | |
| | | | CCTCCCATAAAT | | | |
| | | | C | | | |
| S21220 | SEQ ID NO: | S21220A1FM | TTTCTTGAATGT | 133 | FAM | A |
| | 132 | | AGGTTCCT | | | |
| S21220 | | S21220A2TT | TCTTGAATGTGG | 134 | TET | G |
| | | | GTTCCT | | | |
| S21220 | | S21220F1 | TCTTGGGCGTGC | 135 | | |
| | | | ATTTTATGAA | | | |
| S21220 | | S21220R1 | GCAGTCACTCAA | 136 | | |
| | | | AGCTCTGTG | | | |
| S21224 | SEQ ID NO: | S21224A1FM | AATGCATCCAGC | 138 | FAM | A |
| | 137 | | AGTTCC | | | |
| S21224 | | S21224A2TT | CATCCAGCGGTT | 139 | TET | G |
| | | | CCAA | | | |
| S21224 | | S21224F1 | CCAAGCAAGTTT | 140 | | |
| | | | GAAGCAGCA | | | |
| S21224 | | S21224R1 | GGATTGGTTATT | 141 | | |
| | | | GATGTGTTCGTT | | | |
| | | | C | | | |
| S21227 | SEQ ID NO: | S21227A1FM | TTCCAGCGCAAG | 143 | FAM | A |
| | 142 | | ATGG | | | |
| S21227 | | S21227A2TT | CAGCGCACGAT | 144 | TET | C |
| | | | GGG | | | |
| S21227 | | S21227F1 | GGAGAGATATC | 145 | | |
| | | | CTGCGGTAGAC | | | |
| S21227 | | S21227R1 | GGTGCTCACGT | 146 | | |
| | | | GCTCCAT | | | |
| S21228 | SEQ ID NO: | S21228A1FM | CCTTGAGAGCCA | 148 | FAM | A |
| | 147 | | AATGAAA | | | |
| S21228 | | S21228A2TT | CCTTGAGAGCCG | 149 | TET | G |
| | | | AATGAA | | | |
| S21228 | | S21228F1 | CATTCCATAGGC | 150 | | |
| | | | AATAGCTTCCAG | | | |
| S21228 | | S21228R1 | AAGTGGGTGTC | 151 | | |
| | | | TCCTATTACCAT | | | |
| | | | AC | | | |
| S21229 | SEQ ID NO: | S21229A1FM | CAATGATGTAGA | 153 | FAM | C |
| | 152 | | TTGTCACTAT | | | |
| S21229 | | S21229A2TT | TGATGTAGATTG | 154 | TET | G |
| | | | TGACTATCA | | | |
| S21229 | | S21229F1 | ATCCTGTTTTCT | 155 | | |
| | | | AGGGCATGACC | | | |
| | | | AA | | | |
| S21229 | | S21229R1 | ACAACTTGTCCA | 156 | | |
| | | | AGGTCCACTCAA | | | |
| S21231 | SEQ ID NO: | S21231A1FM | TTGGGTGGATG | 158 | FAM | G |
| | 157 | | CGAAGTA | | | |
| S21231 | | S21231A2TT | TTGGGTGGATG | 159 | TET | C |
| | | | GGAAGTAC | | | |
| S21231 | | S21231F1 | GAACCCGTGCC | 160 | | |
| | | | GTAATCC | | | |
| S21231 | | S21231R1 | ACAGTATGAGT | 161 | | |
| | | | GGAAGGTATGA | | | |
| | | | GG | | | |
| S21232 | SEQ ID NO: | S21232A1FM | CGAATCTGCTGT | 163 | FAM | A |
| | 162 | | GATTGTT | | | |
| S21232 | | S21232A2TT | AATCTGCTGTGG | 164 | TET | G |
| | | | TTGTTGA | | | |
| S21232 | | S21232F1 | GAAACAGCTTGT | 165 | | |
| | | | GGTTGAAGAGA | | | |
| | | | A | | | |
| S21232 | | S21232R1 | CGGAGCCATTTG | 166 | | |
| | | | ATTCATGTCA | | | |
| S21233 | SEQ ID NO: | S21233A1FM | TCTGTTTCTATTA | 168 | FAM | A |
| | 167 | | ACATTTCA | | | |
| S21233 | | S21233A2TT | TCTGTTTCTATTT | 169 | TET | T |
| | | | ACATTTCA | | | |
| S21233 | | S21233F1 | GCACCACACTTC | 170 | | |
| | | | CACTCTCA | | | |
| S21233 | | S21233R1 | TGCTGCCATTGC | 171 | | |
| | | | TCTTCTTC | | | |
| S21234 | SEQ ID NO: | S21234A1FM | TGGTTTAACTCC | 173 | FAM | G |
| | 172 | | GTACTAA | | | |
| S21234 | | S21234A2TT | TTTGGTTTAACT | 174 | TET | A |
| | | | CTGTACTAA | | | |
| S21234 | | S21234F1 | TTGGTGAAGGA | 175 | | |
| | | | TACTCCCCATTC | | | |
| S21234 | | S21234R1 | GTGGAAAAGAT | 176 | | |
| | | | TATTGTGGCCTG | | | |
| | | | AA | | | |
| S21235 | SEQ ID NO: | S21235A1FM | AGCGAGTTACTC | 178 | FAM | G |
| | 177 | | TGGATACC | | | |
| S21235 | | S21235A2TT | AGCGAGTTACTT | 179 | TET | A |
| | | | TGGATACC | | | |
| S21235 | | S21235F1 | ACCAACGGATTG | 180 | | |
| | | | GCATGCTTC | | | |
| S21235 | | S21235R1 | TGAATTGGCAAA | 181 | | |
| | | | TGATCTTGAGGT | | | |
| | | | T | | | |
| S21237 | SEQ ID NO: | S21237A1FM | AGCCACCCTCTC | 183 | FAM | G |
| | 182 | | AAGAA | | | |
| S21237 | | S21237A2TT | AGCCACCCTTTC | 184 | TET | A |
| | | | AAGAAG | | | |
| S21237 | | S21237F1 | CAGAGGAGGAG | 185 | | |
| | | | AGTCCAAGTGT | | | |
| S21237 | | S21237R1 | TGTTGGAGGTG | 186 | | |
| | | | TTGGTTGTGAT | | | |
| S21238 | SEQ ID NO: | S21238A1FM | TTAAATGCCTCA | 188 | FAM | A |
| | 187 | | CAACAC | | | |
| S21238 | | S21238A2TT | ATGCCTCGCAAC | 189 | TET | G |
| | | | ACTG | | | |
| S21238 | | S21238F1 | CCATCAATTTGA | 190 | | |
| | | | ATCACGGGTTTG | | | |
| | | | C | | | |
| S21238 | | S21238R1 | GTTGGACATCGA | 191 | | |
| | | | TTGATACAGACA | | | |
| S21239 | SEQ ID NO: | S21239A1FM | TGACGAACTTGA | 193 | FAM | A |
| | 192 | | AGGTGA | | | |
| S21239 | | S21239A2TT | CGAACTTGAGG | 194 | TET | G |
| | | | GTGAAGA | | | |
| S21239 | | S21239F1 | GGTGAGGAAGA | 195 | | |
| | | | AGTCGAAGATG | | | |
| | | | T | | | |
| S21239 | | S21239R1 | TTCGCCTCTCCTT | 196 | | |
| | | | CACAAC | | | |
| S21242 | SEQ ID NO: | S21242A1FM | AACCCAAGTCAA | 198 | FAM | G |
| | 197 | | CTACTATA | | | |
| S21242 | | S21242A2TT | TGAACCCAAGTC | 199 | TET | A |
| | | | AATTACTAT | | | |
| S21242 | | S21242F1 | TCTCCCTTTGGC | 200 | | |
| | | | TGAATCCAC | | | |
| S21242 | | S21242R1 | CACCAGCATAAC | 201 | | |
| | | | CAGCTTTACAA | | | |
| S21243 | SEQ ID NO: | S21243A1FM | TGGTAAGAGAA | 203 | FAM | A |
| | 202 | | GAGATATCGC | | | |
| S21243 | | S21243A2TT | TGGTAAGAGAA | 204 | TET | G |
| | | | GAGGTATCGC | | | |
| S21243 | | S21243F1 | GACAGAGAAAG | 205 | | |
| | | | AGACAGCACCAT | | | |
| | | | T | | | |
| S21243 | | S21243R1 | AGCCGCGCCTCT | 206 | | |
| | | | AACTTGTAG | | | |
| S21244 | SEQ ID NO: | S21244A1FM | TTGCCTTTCTAA | 208 | FAM | A |
| | 207 | | CTCTAAG | | | |
| S21244 | | S21244A2TT | TGCCTTTCTGAC | 209 | TET | G |
| | | | TCTAAG | | | |
| S21244 | | S21244F1 | GCAAGGAGGGC | 210 | | |
| | | | ATTTGGAA | | | |
| S21244 | | S21244R1 | CCTGAATCGTCC | 211 | | |
| | | | CTGAATCATC | | | |
| S2144 | SEQ ID NO: | S2144A1FM | CAATGAAGATG | 213 | FAM | G |
| | 212 | | GGAAACAGT | | | |
| S2144 | | S2144A2TT | TACAATGAAGAT | 214 | TET | A |
| | | | GGGAAATAGT | | | |
| S2144 | | S2144F1 | GCGTATGAATG | 215 | | |
| | | | GTGTATGCGA | | | |
| S2144 | | S2144R1 | CACTGCCAACCA | 216 | | |
| | | | AAATAGTGACTT | | | |
| S2187 | SEQ ID NO: | S2187A1FM | TAAACTTACCTA | 218 | FAM | T |
| | 217 | | TGATGCAC | | | |
| S2187 | | S2187A2TT | CTAAACTTACCT | 219 | TET | A |
| | | | TTGATGCA | | | |
| S2187 | | S2187F1 | ATGGGCTTTGTG | 220 | | |
| | | | AATATCCAACG | | | |
| S2187 | | S2187R1 | CCTCTCAATTTG | 221 | | |
| | | | AAGTATGCTCCA | | | |
| S256 | SEQ ID NO: | S256A1FM | CACCGTATCGAC | 223 | FAM | G |
| | 222 | | GTGT | | | |
| S256 | | S256A2TT | CACCGTATTGAC | 224 | TET | A |
| | | | GTGTG | | | |
| S256 | | S256F1 | CAGAGAGAATC | 225 | | |
| | | | GCGTCCGA | | | |
| S256 | | S256R1 | TTGACCGCGTGT | 226 | | |
| | | | CCGA | | | |

### Example 4: Wide Crossing, Embryo Rescue & Introgression of Resistance Conferring Intervals into Glycine max Lines

Embryo rescue will be performed and chemical treatment will be applied in order to generate amphidiploid shoots. If the amphidiploid plants are fertile they will be used to backcross with G. max. Backcrossing with G. max and subsequent embryo rescue will need to be performed for several generations in order to gradually eliminate the perennial Glycine chromosomes.

Wide crosses: Elite Syngenta soybean lines (RM 3.7 to 4.8) will be used as the females (pollen recipients) and multiple accessions of Glycine canescens will be used as the males or pollen donors. Flowers will be collected from the glycine plant containing anthers at the proper developmental stage. This will include new, fully-opened, brightly colored flowers holding anthers with mature pollen that appears as loose, yellow dust. These flowers will be removed from the glycine plant and taken to the soybean plant for pollination. Pollen from the Glycine plants will be used within 30 minutes of flower removal. Soybean flower buds will be selected for pollination when they are larger in size compared to an immature bud, when the sepals of the soybean blossoms are lighter in color, and the petals are just beginning to appear. The sepals will be detached from the flower bud to expose the outer set of petals which will then be removed from the flower to expose the ring of stamens surrounding the pistil. Using 1 male flower, the anthers will be exposed, and pollen grains will be gently dusted onto the stigma of the soybean flower. Starting the day after pollination a hormone mixture will be sprayed onto the pollinated flower and eventual developing F1 pod once every day until harvest. The pollinated flower or pod will be saturated with a light mist of the hormone mixture (containing 100 mg GA3, 25 mg NAA and 5 mg kinetin/L distilled water), to aid in the retention of the developing pod and in increased pod growth.

Harvest: Pods from wide crosses will be harvested at approximately 14 to 16 days post pollination. Before selecting an individual pod to harvest, it will be verified that the sepals are removed and the seed size is as expected for a wide cross. Pods will be collected and counted according to wide cross combinations to determine crossing success. The wide cross pods are expected to contain 1 to 3 seeds.

Embryo rescue: Harvested pods will be sterilized by first rinsing with 70% EtOH for 2 to 3 minutes and then placing in 10% Clorox bleach for an additional 30 minutes on a platform shaker at approximately 130 RPM. After rinsing the pods multiple times with sterile water to remove any residual bleach, embryo isolation will be performed. If this is not started immediately following pod sterilization, pods will be stored at 4° C. for up to 24 hours prior to embryo isolation. Next, in a laminar flow hood, individual pods will be placed in a sterile petri dish and opened using a scalpel and forceps. An incision will be made along the length of the wide cross pod away from the seed, to expose the seed. The seed will be removed from the pod and placed in a sterile petri dish under the dissection microscope. Holding the side of the seed away from the embryo, with hilum facing up, the seed coat will be removed from the side of the seed containing the embryo. After peeling off the membrane surrounding the embryo, the embryo will be pushed up from its bottom side. Embryos should be past the globular developmental stage and preferably past the early heart developmental stage (middle to late heart stage, cotyledon stage and early maturation stage embryos are desired). Isolated embryos will be transferred to embryo rescue medium such as Soy ER1-1. Embryos will be treated to induce chromosome doubling at this time. (See below). Isolated embryos will be maintained on embryo rescue medium for 21 to 30 days at 24° C. No callus induction stage will occur in this protocol. Shoots will develop directly from the embryos.

Chromosome doubling treatments: Colchicine of trifluralin will be used to induce chromosome doubling. Ideally, late heart stage wide cross embryos (or larger) will be chemically treated to induce chromosome doubling at any time from immediately following isolation up to 1 week post isolation. The doubling agent will be mixed in either solid or liquid medium and applied for several hours or up to a few days. Trifluralin will be used at a concentration of 10-40 uM in either solid or liquid media. Use of trifluarin will reduce the colchicine requirement. Colchicine will be used at a concentration of 0.4-1 mg/ml in either solid or liquid media. Following the chemical treatment, the embryos will be transferred to fresh embryo rescue medium.

Shoot regeneration: Developing embryos will be transferred from rescue medium to germination medium such as Soy ER GSMv2 for approximately 3 to 5 weeks in the light at 24° C. Alternatively, developing embryos can be transferred from rescue medium to elongation medium such as Soy E1 0 No TCV for approximately 3 to 5 weeks in the light at 24° C. Developing shoots will be transferred from media plates to Phytocons containing either germination or elongation medium for further shoot development. Established shoots will be moved to soil.

Ploidy Analysis: Ploidy analysis will be conducted using a flow cytometer. Leaf tissue for ploidy analysis will be collected from small shoots either in culture or after establishment in soil. Tissue will be collected on dry ice and stored at −80° C. until analysis or collected on wet ice and analyzed the same day. A sample size of 0.5 cm² will be sufficient. Samples will be prepared according to the instructions in the Sysmex kit. Each sample set will contain an untreated F1 plant (not treated to induce chromosome doubling) as a control.

Harvest—Pods will be harvested at 14 to 16 days after pollination.

Embryo rescue—Since the disclosed embryo rescue protocol involves direct shoot regeneration from embryos, rather than regeneration through embryogenesis, plant recovery will be expedited with shoot recovery in approximately 2-3 months.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

1.-17. (canceled)

18. A method of producing a soybean plant having increased resistance to ASR, the method comprising the steps of:
a) detecting in a population of soybean plants, the presence of an ASR resistance allele associated with increased ASR resistance, wherein the ASR resistance allele is linked to a chromosomal interval comprising SEQ ID NO: 1 or a portion thereof, and wherein the ASR resistance allele is any of the favorable ASR resistance alleles of Table 1 or a linked allele located within 20 cM, 10 cM, 5 cM, 1 cM, 0.5 cM thereof; and
b) selecting from the population of soybean plants, a selected soybean plant based on the presence of the ASR resistance allele of a) and exhibiting increased ASR resistance as compared to a control plant lacking said allele.

19. The method of claim 18, further comprising, assaying the selected soybean plant for resistance to ASR.

20.-23. (canceled)

24. The method of any one of claims 18-19, wherein said chromosomal interval, or portion thereof, associated with the ASR resistance allele was introgressed into the population of soybean plants from a soybean plant comprising the chromosomal interval, or portion thereof, and further comprising the ASR resistance allele.

25. A method of producing a soybean plant with enhanced ASR resistance comprising:
a) crossing a first soybean plant comprising an ASR resistance allele with a second soybean plant of a different genotype to produce one or more progeny plants; and
b) selecting a progeny soybean plant based on the presence of said ASR resistance allele at a locus genetically linked to a chromosomal interval comprising SEQ ID NO: 1 or a portion thereof,
wherein said ASR resistance allele confers enhanced resistance to ASR compared to a plant lacking said allele, and wherein the ASR resistance allele is any of the favorable ASR resistance alleles of Table 1 or a linked allele located within 20 cM, 10 cM, 5 cM, 1 cM, 0.5 cM thereof.

26. The method of claim 25, wherein said chromosomal interval is flanked by SNP marker 1850 and SNP marker 3656 of Table 1.

27. The method of claim 26, wherein said chromosomal interval is flanked by SNP marker 3115 and SNP marker 3347 of Table 1.

28. A method of identifying or selecting an ASR resistant soybean plant having an ASR resistance allele derived from Glycine canescens, the method comprising the steps of:
a. isolating nucleic acid from a soybean plant; and
b. detecting in the nucleic acid the presence of a molecular marker that associates with increased ASR resistance wherein the molecular marker is located within 20 cM, 10 cM, 5 cM, 1 cM, or 0.5 cM of a favorable marker as described in Table 1; and
c. identifying or selecting a soybean plant having the ASR resistance allele derived from Glycine canescens on the basis of the molecular marker detected in b).

28. The method of claim 25, wherein producing the progeny plant having increased ASR resistance comprises use of introgression of a chromosomal interval comprising SEQ ID NO: 1, or a portion thereof, from a wild Glycine species and/or chemically induced chromosome doubling.

29. A method of producing a Glycine soybean plant having increased resistance to Asian soybean rust (ASR), the method comprising the steps of:
a) wide crossing a Glycine max plant with a ASR resistant Glycine canescens plant comprising in its genome a chromosomal interval comprising SEQ ID NO: 1 or a portion thereof;
b) generating soybean pods from the cross of a);
c) isolating embryos from the pods of b) and placing said embryos onto embryo rescue medium, wherein there is no callus induction;
d) analyzing genomic nucleic acid isolated from the embryo of c) for the presence of an allele that associates with increased ASR resistance and is linked with or located within the chromosome interval comprising SEQ ID NO: 1 or the portion thereof, or a molecular marker linked thereto and located within 20 cM, 10 cM, 5 cM, 4 cM, 3 cM, 2 cM, 1 cM, or 0.5 cM thereof;
e) transferring the embryos of d) onto germination medium or elongation medium and collecting shoots from said embryos;
f) growing the collected shoots of e) on germination or elongation medium; and
g) transferring established shoots of f) to soil, thereby producing a soybean plant having increased resistance to ASR.

30. The method of claim 29, wherein the embryos and/or shoots are treated with a chromosome doubling agent in any one of steps e)-h) to create amphidiploid plants capable of being backcrossed with a domestic annual Glycine cultivar to produce at least one backcross generation (BC1), wherein the chromosome doubling agent is either colchicine or trifluralin.
